(19) United States
(12) Patent Application Publication
(10) Pub. No.: US 2003/0171368 A1
(43) Pub. Date: Sep. 11, 2003

(54) Pyrimidinonesulfamoylureas`

(76) Inventors: Werner Seitz, Planckstadt (DE); Andreas Kling, Mannheim (DE); Herve Geneste, Neuhofen (DE); Thomas Subkowski, Ladenburg (DE); Claudia Graef, Mannheim (DE); Wilfried Hornberger, Neustadt (DE)

Correspondence Address: KEIL & WEINKAUF, 1350 CONNECTICUT AVENUE, N.W., WASHINGTON, DC, 20036, US

(21) Appl. No.: 10/066,624

(22) Filed: Feb. 6, 2002

(51) Int. Cl. A61K 31/54; A61K 31/53; A61K 31/513; A61K 31/522; C07D 417/02; C07D 473/00; C07D 487/02; C 07D 4 3/02

(52) U.S. Cl. 514/227.2; 514/243; 514/263.31; 514/269; 544/54; 544/182; 544/277; 544/296; 544/316; 544/314

(57) ABSTRACT

The disclosure is directed to compounds of the formula (I)

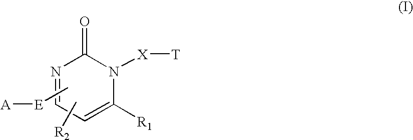

wherein the radicals have the meanings described in the disclosure, to the preparation of the compounds and to the use of the compounds to find the integrin receptors.

The invention relates to novel compounds which bind to integrin receptors, and to their preparation and use.

Integrins are cell surface glycoprotein receptors which mediate interactions between similar and different cells, and between cells and extracellular matrix proteins. They are involved in physiological processes, such as, for example, embryogenesis, hemostasis, wound-healing, immune response and formation/maintenance of the tissue architecture.

Disorders in the gene expression of cell adhesion molecules and functional disorders of the receptors can contribute to the pathogenesis of many diseases, such as, for example, tumors, thromboembolic events, cardiovascular diseases, pulmonary diseases, diseases of the CNS, of the kidney, of the gastrointestinal tract or inflammation.

Integrins are heterodimers composed of one α- and one β-transmembrane subunit in each case, which are noncovalently bonded.

Up to now, 16 different α- and 8 different β-subunits and 22 different combinations have been identified.

Integrin αᵥβ₃, also called vitronectin receptor, mediates adhesion to a large number of ligands—plasma proteins, extracellular matrix proteins, cell surface proteins, of which the majority contain the amino acid sequence RGD (Cell, 1986, 44, 517-518; Science 1987, 238, 491-497), such as, for example, vitronectin, fibrinogen, fibronectin, von Willebrand factor, thrombospondin, osteopontin, laminin, collagen, thrombin, tenascin, MMP-2, bone sialoprotein II, various viral, fungal, parasitic and bacterial proteins, natural integrin antagonists such as disintegrins, neurotoxins—mambin—and leech proteins—decorsin, ornatin—and some non-RGD ligands, such as, for example, Cyr-61 and PECAM-1 (L. Piali, J. Cell Biol. 1995, 130, 451-460; Buckley, J. Cell Science 1996, 109, 437-445, J. Biol. Chem. 1998, 273, 3090-3096).

A number of integrin receptors show cross-reactivity with ligands which contain the RGD motif. Thus integrin α_{IIb}β₃, also called platelet fibrinogen receptor, recognizes fibronectin, vitronectin, thrombospondin, von Willebrand factor and fibrinogen.

Integrin αᵥβ₃ is expressed, inter alia, on endothelial cells, blood platelets, monocytes/macrophages, smooth muscle cells, some B cells, fibroblasts, osteoclasts and various tumor cells, such as, for example, melanoma, glioblastoma, lung, breast, prostate and bladder carcinoma, osteosarcoma or neuroblastoma.

Increased expression is observed under various pathological conditions, such as, for example, in the prothrombotic state, in the case of vascular injury, tumor growth or metastasis or reperfusion and on activated cells, in particular on endothelial cells, smooth muscle cells or macrophages.

Involvement of integrin αᵥβ₃ has been demonstrated, inter alia, in the following syndromes:

cardiovascular diseases such as atherosclerosis, restenenosis after vascular injury, and angioplasty (neointima formation, smooth muscle cell migration and proliferation) (J. Vasc. Surg. 1994, 19, 125-134; Circulation 1994, 90, 2203-2206),

acute kidney failure (Kidney Int. 1994, 46, 1050-1058; Proc. Natl. Acad. Sci. 1993, 90, 5700-5704; Kidney Int. 1995, 48, 1375-1385),

angiogenesis-associated microangiopathies such as, for example, diabetic retinopathy or rheumatoid arthritis (Ann. Rev. Physiol 1987, 49, 453-464; Int. Ophthalmol. 1987, 11, 41-50; Cell 1994, 79, 1157-1164; J. Biol. Chem. 1992, 267, 10931-10934),

arterial thrombosis,

stroke (Phase II studies with ReoPro, Centocor Inc., 8th annual European Stroke Meeting),

cancers, such as, for example, in tumor metastasis or in tumor growth (tumor-induced angiogenesis) (Cell 1991, 64, 327-336; Nature 1989, 339, 58-61; Science 1995, 270, 1500-1502),

osteoporosis (bone resorption after proliferation, chemotaxis and adhesion of osteoclasts to bone matrix) (FASEB J. 1993, 7, 1475-1482; Exp. Cell Res. 1991, 195, 368-375, Cell 1991, 64, 327-336),

high blood pressure (Am. J. Physiol. 1998, 275, H1449-H1454),

psoriasis (Am. J. Pathol. 1995, 147, 1661-1667),

hyperparathyroidism,

Paget's disease (J. Clin. Endocrinol. Metab. 1996, 81, 1810-1820),

malignant hypercalcemia (Cancer Res. 1998, 58, 1930-1935),

metastatic osteolytic lesions (Am. J. Pathol. 1997, 150, 1383-1393),

pathogenic protein (e.g. HIV-1 tat) induced processes (e.g. angiogenesis, Kaposi's sarcoma) (Blood 1999, 94, 663-672)

inflammation (J. Allergy Clin. Immunol. 1998, 102, 376-381),

cardiac insufficiency, CHF, and in

antiviral, antiparasitic, antifungal or antibacterial therapy and prophylaxis (adhesion and internalization) (J. Infect. Dis. 1999, 180, 156-166; J. Virology 1995, 69, 2664-2666; Cell 1993, 73, 309-319).

On account of its key role, pharmaceutical preparations which contain low molecular weight integrin αᵥβ₃ ligands are of high therapeutic and diagnostic benefit, inter alia, in the indications mentioned.

Advantageous αᵥβ₃-integrin receptor ligands bind to the integrin αᵥβ₃ receptor with an increased affinity.

Particularly advantageous αᵥβ₃-integrin receptor ligands additionally have, compared with integrin αᵥβ₃, increased selectivity and, relative to integrin αᵥβ₃ are less active by at least a factor of 10, preferably by at least a factor of 100.

For a large number of compounds, such as anti-αᵥβ₃ monoclonal antibodies, peptides which contain the RGD-binding sequence, natural, RGD-containing proteins (e.g. disintegrins) and low molecular weight compounds, an integrin αᵥβ₃ antagonistic action has been shown and a positive in vivo effect demonstrated (FEBS Letts 1991, 291, 50-54; J. Biol. Chem. 1990, 265, 12267-12271; J. Biol. Chem. 1994, 269, 20233-20238; J. Cell Biol. 1993, 51, 206-218; J. Biol. Chem. 1987, 262, 17703-17711; Bioorg. Med. Chem. 1998, 6, 1185-1208).

In WO 99/30713, 1,3-disubstituted tetrahydropyrimidin-2(1H)-one derivatives and piperidin-2-one derivatives, in the specification WO 99/31099 1,3-disubstituted imidazolin-2-one derivatives, in the specification WO 98/35949 2,6-disubstituted 2H-1,4-benzoxazin-3(4H)-one derivatives, in the specifications WO 98/00395 and WO 97/23451 0-substituted tyrosine derivatives, in EP 710657 and EP 74/1333,5-disubstituted 1,3-oxazolidin-2-one and in the specification WO 97/37655 isoindoles are described as antagonists of the αᵥβ₃-integrin receptor.

WO 00/61551 describes substituted pyrimidinone derivatives as integrin ligands which already have good activities and selectivities. Nevertheless, the need furthermore exists to improve the activities and selectivities and also the pharmacokinetic properties of the integrin ligands further.

The object of the invention is therefore to make available novel integrin receptor ligands having advantageous properties, such as improved activities, selectivities and pharmacokinetic properties.

The object is achieved by compounds of the general formula (I)

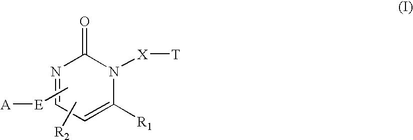

where the radicals have the following meaning:

T=CO₂H, a radical hydrolyzable to CO₂H or a radical isosteric to CO₂H, such as described, for example, in “The Practice of Medicinal Chemistry”, ed. C. G. Wermuth, Academic Press 1996, pp.215-221,

X=—(CR_{X}¹ R_{X}²)ₐ—(G_{X})ₑ—(CR_{X}³R_{X}⁴)_{b}—W_{X}—(CR_{X}⁵R_{X}⁶)_{c}—(Y_{X})_{f}—(CR_{X}⁷R_{X}⁸)_{d}—where

a, b, c, d independently of one another are 0, 1, 2 or 3,

e, f independently of one another are 0 or 1,

the sum of a, b, c, d, e and f is ≦10,

R_{X}¹, R_{X}², R_{X}³, R_{X}⁴, R_{X}⁵, R_{X}⁶, R_{X}⁷, R_{X}⁸ independently of one another are hydrogen, halogen, a hydroxyl group, a branched or unbranched, optionally substituted C₁-C₆-alkyl, C₂-C₆-alkenyl, C₂-C₆-alkynyl or alkylenecycloalkyl radical, a radical —(CH₂)ᵣ—(Y_{X})_{y}—R_{X}⁹, an optionally substituted C₃-C₇-cycloalkyl, aryl, arylalkyl, hetaryl or hetarylalkyl radical, or independently of one another in each case two radicals R_{X}¹ and R_{X}² or R_{X}³ and R_{X}⁴ or R_{X}⁵ and R_{X}⁶ or R⁷ and R_{X}⁸ together are a 3 to 7-membered, optionally substituted, saturated or unsaturated carbo- or heterocycle, which can contain up to three heteroatoms from the group consisting of O, N and S,

R_{X}⁹ is hydrogen, a hydroxyl group, CN, halogen, a branched or unbranched, optionally substituted C₁-C₆-alkyl, aryl or alkylenearyl radical, a primary or optionally secondary or tertiary substituted amino radical, a C₂-C₆-alkynyl or C₂-C₆-alkenyl radical optionally substituted by C₁-C₄-alkyl or aryl, a C₅-C₁₂-bicycloalkyl or C₈-C₂₀-tricycloalkyl radical, or a 3-to 6-membered, saturated or unsaturated heterocycle substituted by up to three identical or different radicals, which can contain up to three different or identical heteroatoms O, N, S, or a C₃-C₇-cycloalkyl, aryl or heteroaryl radical, where two radicals together can be a fused, saturated, unsaturated or aromatic carbocycle or heterocycle which can contain up to three different or identical heteroatoms O, N, S, and the cycle can optionally be substituted or a further, optionally substituted, saturated, unsaturated or aromatic cycle can be fused to this cycle,

r=0-4

G_{X} and Y_{X} independently of one another are CO, CO—NR_{X}¹⁰, NR_{X}¹⁰CO, S, SO, SO₂, SO₂NR_{X}¹⁰, NR_{X}¹⁰SO₂, CS, CS—NR_{X}¹⁰, NR_{X}¹⁰—CS, CS—O, O—CS, CO—O, O—CO, O, ethynyl, CR_{X}¹¹—O—CR_{X}, C(═CR_{X}¹¹R_{X}¹²), CR_{X}¹¹═CR_{X}¹², CR_{X}¹¹(OR_{X}¹³)—CR_{X}¹², CR_{X}¹¹—CR_{X}¹²(OR_{X}¹³)—,

R_{X}¹⁰ is hydrogen, a branched or unbranched, optionally substituted C₁-C₆-alkyl, alkylenearyl, alkylenealkynyl, hetaryl or alkylenehetaryl radical, an optionally substituted C₃-C₇-cycloalkyl radical, CO—R_{X}¹⁴, COOR_{X}¹⁴, SO₂—R_{X}¹⁴,

R_{X}¹¹, R_{X}¹² independently of one another are hydrogen, a hydroxyl group, a branched or unbranched, optionally substituted C₁-C₆-alkyl, C₁-C₄-alkoxy, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,

R_{X}¹³ is hydrogen, a branched or unbranched, optionally substituted C₁-C₆-alkyl, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,

R_{X}¹⁴ is hydrogen, a hydroxyl group, a branched or unbranched, optionally substituted C₁-C₆-alkyl, aryl, heterocyclyl, heteroaryl, C₃₋₇-cycloalkyl, alkylenecycloalkyl, alkylenearyl, alkyleneheterocyclyl, alkyleneheteroaryl, C₂₋₆-alkenyl, C₂₋₆-alkynyl, C₁₋₄-alkoxy-C₁₋₅-alkyl radical, where in saturated carbocyclic radicals 1-2 atoms can also be replaced by heteroatoms, preferably N, O, or S, and up to 2 double bonds can be contained.

y=0, 1

W_{X}:

is —(CR_{w}⁴R_{w}⁵)_{w}—N R_{w}¹SO₂NR_{w}²R_{w}³

where w=0-3

R_{w}¹ is hydrogen, a branched or unbranched, optionally substituted C₁-C₆-alkyl, C₁-C₆-alkoxyalkyl, alkylenearyl, alkylenealkynyl, hetaryl, CO—C₁-C₆-alkyl, CO—O—C₁-C₆-alkyl or SO₂—C₁-C₆-alkyl radical or an optionally substituted C₃-C₇-cycloalkyl, CO—O-alkylenearyl, CO-alkylenearyl, CO-aryl, SO₂-aryl, CO-hetaryl or SO₂-alkylenearyl radical,

R_{w}², R_{w}³ independently of one another are hydrogen, a hydroxyl group, a branched or unbranched, optionally substituted C₁-C₈-alkyl, C₂-C₆-alkenyl, C₂-C₆-alkynyl or alkylenecycloalkyl radical, an optionally substituted C₃-C₈-cycloalkyl, aryl, arylalkyl, hetaryl or hetarylalkyl radical, or independently of one another both radicals R_{w}² and R_{w}³ together are a 3- to 7-membered, optionally substituted, saturated or unsaturated carbo- or heterocycle, which can contain up to three heteroatoms from the group consisting of O, N and S,

R_{w}⁴, R_{w}⁵ independently of one another are C₁₋₈-alkyl, halogen, OH, C₁₋₈-alkoxy,

R₁, R₂ independently of one another are hydrogen, halogen, CF₃, CN, NO₂, branched or unbranched C₁₋₈-alkyl, C₃₋₇-cycloalkyl, alkylcycloalkyl, where in each case 1-3 atoms in the cycloalkyl moiety can be replaced by N, O or S and up to 2 double bonds can be contained,

aryl, alkylenearyl, hetaryl, alkylenehetaryl, C₂₋₆-alkenyl, C₃₋₆-alkynyl, C₀₋₄-alkyl-OR₃, C₀₋₄-alkyl-SR₃, SO—R₃, SO₂—R₃, CO₄(CO)OR₃, O(CO)R₃, O(CO)NR₄R₅, C₀₋₄-alkyl-SO₂NR₄R₅, C₀₋₄—(CO)NR₄R₅, CO₄-alkyl-NR₄R₅, CO—R₃,

or R₁ and R₂ together are a 3- to 9-membered optionally substituted cyclic or polycyclic system, which can contain 0-4 heteroatoms from the group consisting of O, N and S,

R₃ is H, or C₁₋₈-alkyl, aryl, heterocyclyl, heteroaryl, C₃₋₇-cycloalkyl, alkylenecycloalkyl, alkylenearyl, alkyleneheterocyclyl, alkyleneheteroaryl, C₂₋₆-alkenyl, C₂₋₆-alkynyl, C₁₋₄-alkoxy-C₁₋₅-alkylene, mono- and bisalkylaminoalkylene, acylaminoalkylene, each of which is branched or straight-chain and optionally substituted by halogen, OH, alkoxy, CN, COOH, COOC₁₋₄-alkyl, where in saturated carbocyclic radicals 1-2 atoms can also be replaced by heteroatoms, preferably N, O, or S, and up to 2 double bonds can be contained,

R₄, R₅ is H, C₀₋₁₈-alkyl, aryl, heterocyclyl, heteroaryl, C₃₋₇-cycloalkyl, alkylenecycloalkyl, alkylenearyl, alkyleneheterocyclyl, alkyleneheteroaryl, C₂₋₆-alkenyl, C₂₋₆-alkynyl, C₁₋₄-alkoxy-C₁₋₅-alkylene, mono- and bisalkylaminoalkylene, acylaminoalkylene, each of which is branched or straight-chain and optionally substituted by halogen, OH, alkoxy, CN, COOH, COOC₁₋₄-alkyl, where in saturated carbocyclic radicals 1-2 atoms can also be replaced by heteroatoms, preferably N, O, or S, and up to 2 double bonds can be contained,

A is a structural element selected from the group consisting of:

a 4- to 8-membered monocyclic saturated, unsaturated or aromatic hydrocarbon, which can contain up to 4 heteroatoms, selected from the group consisting of O, N and S, where in each case independently of one another the ring nitrogen optionally contained or the carbons can be substituted,

with the proviso that at least one heteroatom, selected from the group consisting of O, N and S, is contained in the structural element A, or

a 9- to 14-membered polycyclic saturated, unsaturated or aromatic hydrocarbon, which can contain up to 6 heteroatoms, selected from the group consisting of N, O and S, where in each case independently of one another the ring nitrogen optionally contained or the carbons can be substituted,

with the proviso that at least one heteroatom, selected from the group consisting of O, N and S, is contained in the structural element A,

a radical

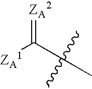

where

Z_{A}¹ is oxygen, sulfur or optionally substituted nitrogen, and

Z_{A}² is optionally substituted nitrogen, oxygen or sulfur, preferably nitrogen,

or a radical

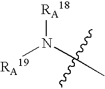

where

R_{A}¹⁸, R_{A}¹⁹

independently of one another are hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- or bisalkylaminoalkylene or acylaminoalkylene radical or an optionally substituted aryl, heterocycloalkyl, heterocycloalkenyl, hetaryl, C₃-C₇-cycloalkyl, C₀-C₄-alkylene-C₃-C₇-cycloalkyl, arylalkyl, C₁-C₄-alkyleneheterocycloalkyl, C₀-C₄-alkyleneheterocycloalkenyl or hetarylalkyl radical, or a radical —SO₂—R₄, —CO—OR₄, —CO—NR₄R₅ or —CO—R₄,

E is a spacer between A and the structural element pyrimidinone having 3-12 bonds.

The compound according to the invention is explained in more detail below.

R₁ is preferably hydrogen, CF₃, CN, branched or unbranched C₁₋₈-alkyl, such as, for example, optionally substituted methyl, ethyl, propyl, 1-methylethyl, butyl, 1-methyl-propyl, 2-methylpropyl, 1,1-dimethylethyl, pentyl, 1-methylbutyl, 2-methylbutyl, 1,2-dimethylpropyl, 1,1-dimethylpropyl, 2,2-dimethylpropyl, 1-ethylpropyl, hexyl, 1-methyl-pentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl or 1-ethyl-2-methylpropyl,

aryl, preferably optionally substituted phenyl, 1-naphthyl or 2-naphthyl,

alkylenearyl, preferably optionally substituted benzyl or ethylenephenyl (homobenzyl),

hetaryl, preferably optionally substituted 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 3-isoxazolyl, 4-isoxazolyl, 5-isoxazolyl, thiadiazolyl, oxadiazolyl or triazinyl or their fused derivatives such as, for example, indazolyl, indolyl, benzothiophenyl, benzofuranyl, indolinyl, benzimidazolyl, benzothiazolyl, benzoxazolyl, quinolinyl or isoquinolinyl,

alkylenehetaryl, preferably optionally substituted —CH₂-2-pyridyl, —CH₂-3-pyridyl, —CH₂-4-pyridyl, —CH₂-2-thienyl, —CH₂-3-thienyl, —CH₂-2-thiazolyl, —CH₂-4-thiazolyl, CH₂-5-thiazolyl, —CH₂—CH₂-2-pyridyl, —CH₂—CH₂-3-pyridyl, —CH₂—CH₂-4-pyridyl, —CH₂—CH₂-2-thienyl, —CH₂—CH₂-3-thienyl, —CH₂—CH₂-2thiazolyl, —CH₂—CH₂-4-thiazolyl or —CH₂—CH₂-5-thiazolyl or

C₂₋₆-alkenyl, such as, for example, optionally substituted vinyl, 2-propenyl, 2-butenyl, 3-butenyl, 1-methyl-2-propenyl, 2-methyl-2-propenyl, 2-pentenyl, 3-pentenyl, 4-pentenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-2-propenyl, 1-ethyl-2-propenyl, 2-hexenyl, 3-hexenyl, 4-hexenyl, 5-hexenyl, 1-methyl-2-pentenyl, 2-methyl-2-pentenyl, 3-methyl-2-pentenyl, 4-methyl-2-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-2-butenyl, 1,2-dimethyl-3-butenyl, 1,3-dimethyl-2-butenyl, 1,3-dimethyl-3-butenyl, 2,2-dimethyl-3-butenyl, 2,3-dimethyl-2-butenyl, 2,3-dimethyl-3-butenyl, 1-ethyl-2-butenyl, 1-ethyl-3-butenyl, 2-ethyl-2-butenyl, 2-ethyl-3-butenyl, 1,1,2-trimethyl-2-propenyl, 1-ethyl-1-methyl-2-propenyl or 1-ethyl-2-methyl-2-propenyl,

C₃₋₆-alkynyl, such as, for example, optionally substituted 2-propynyl, 2-butynyl, 3-butynyl, 1-methyl-2-propynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 1-methyl-2-pentynyl, 1-methyl-2-pentynyl, 1-methyl-3-pentynyl, 1-methyl-4-pentynyl, 2-methyl-3-pentynyl, 2-methyl-4-pentynyl, 3-methyl-4-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 1-ethyl-2-butynyl, 1-ethyl-3-butynyl, 2-ethyl-3-butynyl or 1-ethyl-1-methyl-2-propynyl,

or C₀₋₄-alkyl-OR₃, and in particular hydrogen.

R₂ is preferably hydrogen, Hal, CF₃, CN, branched or unbranched C₁₋₈-alkyl, C₃₋₇-cycloalkyl, alkylcycloalkyl, where in each case 1-2 atoms in the cycloalkyl moiety can be replaced by N, O or S and up to 2 double bonds can be contained, aryl, alkylenearyl, hetaryl, alkylenehetaryl,

in particular branched or unbranched C₁₋₈-alkyl, C₃₋₇-cycloalkyl, alkylcycloalkyl, where in each case 1-2 atoms in the cycloalkyl moiety can be replaced by N, O or S and up to 2 double bonds can be contained, aryl, alkylenearyl, hetaryl, alkylenehetaryl.

In a further preferred embodiment, the radical R₂ is situated in the 5-position and the radical A-E in the 4-position of the pyrimidinone ring.

In a preferred embodiment, Z_{A}¹ in the structural element A is oxygen or nitrogen and Z_{A}² is nitrogen.

In a further preferred embodiment, the structural element A used is a structural element selected from the group consisting of the structural elements of the formulae I_{A}¹ to I_{A}¹⁹,

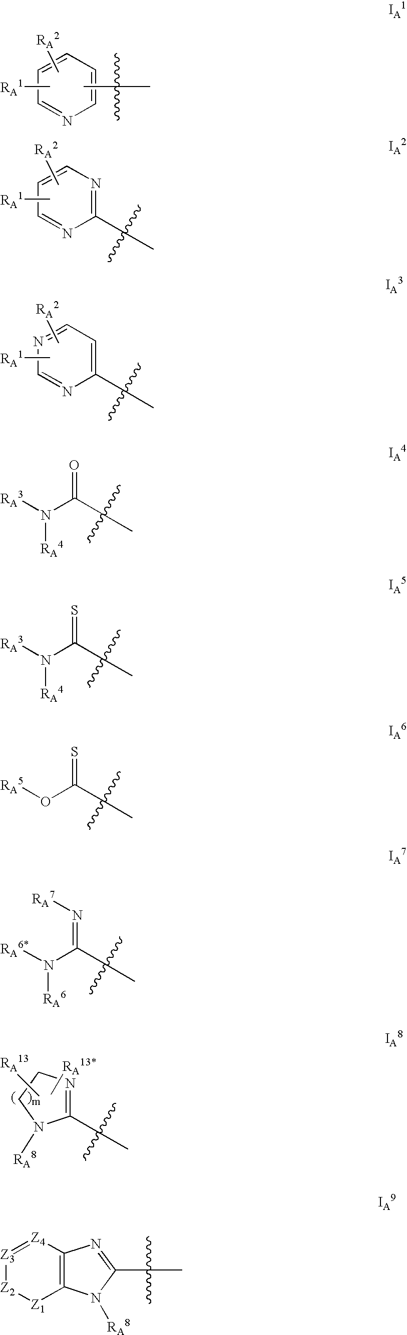

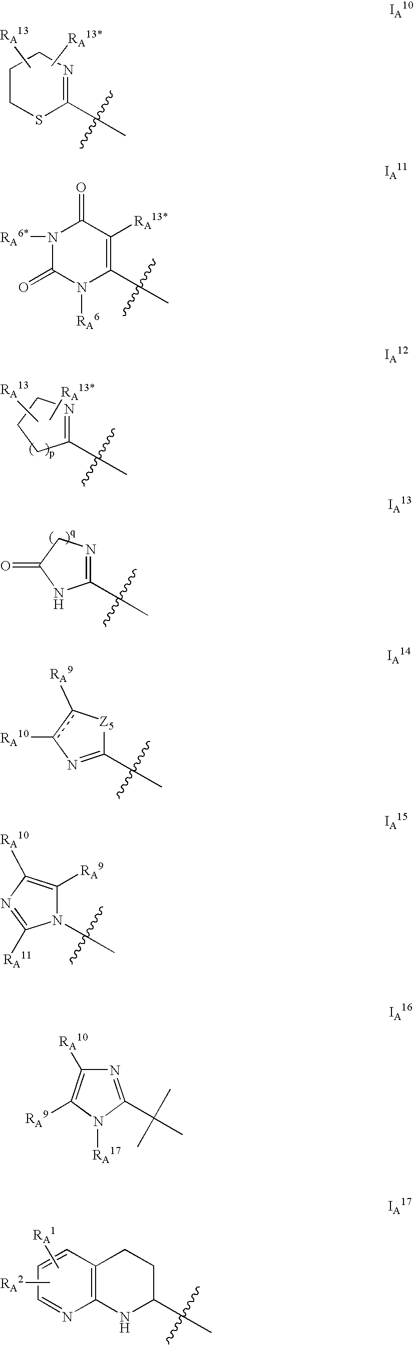

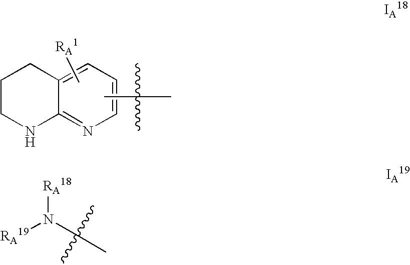

where

m, p, q independently of one another are 1, 2 or 3,

R_{A}¹, R_{A}² independently of one another are hydrogen, CN, halogen, a branched or unbranched, optionally substituted C₁-C₆-alkyl or CO—C₁-C₆-alkyl radical or an optionally substituted aryl, arylalkyl, hetaryl, hetarylalkyl or C₃-C₇-cycloalkyl radical or a radical CO—O—R_{A}¹⁴, O—R_{A}¹⁴, S—R_{A}¹⁴, NR_{A}¹⁵R_{A}¹⁶, CO—NR_{A}¹⁵R_{A}¹⁶ or SO₂NR_{A}¹⁵R_{A}¹⁶ or both radicals R_{A}¹ and R_{A}² together are a fused, optionally substituted, 5- or 6-membered, unsaturated or aromatic carbocycle or heterocycle which can contain up to three heteroatoms, selected from the group consisting of O, N and S,

R_{A}¹³, R_{A}¹³ independently of one another are hydrogen, CN, halogen, a branched or unbranched, optionally substituted C₀-C₆-alkyl radical or an optionally substituted aryl, arylalkyl, hetaryl or C₃-C₇-cycloalkyl radical or a radical CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}¹⁵R_{A}¹⁶,

where

R_{A}¹⁴ is hydrogen, a branched or unbranched, optionally substituted C₁-C₆-alkyl, alkylene-C₁-C₄-alkoxy, C₂-C₆-alkenyl, C₂-C₆-alkynyl or C₁-C₆-alkylene-C₃-C₇-cycloalkyl radical or an optionally substituted C₃-C₇-cycloalkyl, aryl, arylalkyl, hetaryl or hetarylalkyl radical,

R_{A}¹⁵, R_{A}¹⁶ independently of one another are hydrogen, a branched or unbranched, optionally substituted C₁-C₆-alkyl, CO—C₁-C₆-alkyl, SO₂—C₀-C₆-alkyl, CO₀-C₁-C₀₋₆-alkyl, CO—NH—C₁-C₆-alkyl, arylalkyl, COO-alkylenearyl, SO₂-alkylenearyl, CO—NH-alkylenearyl, CO—NH-alkylenehetaryl or hetarylalkyl radical or an optionally substituted C₃-C₇-cycloalkyl, aryl, CO-aryl, CO—NH-aryl, SO₂-aryl, hetaryl, CO—NH-hetaryl, or CO-hetaryl radical,

R_{A}³, R_{A}⁴ independently of one another are hydrogen, —(CH₂)ₙ—(X_{A})ⱼ—R_{A}¹², or both radicals together are a 3- to 8-membered, saturated, unsaturated or aromatic N heterocycle which additionally can contain two further, identical or different heteroatoms O, N, or S, where the cycle can be optionally substituted or a further, optionally substituted, saturated, unsaturated or aromatic cycle can be fused to this cycle,

where

n is 0, 1, 2 or 3,

j is 0 or 1,

X_{A} is —CO—, —CO—N(R_{L}¹)—, —N(R_{L}¹)—CO—, N(R_{L}¹)—CO—N(R_{L}¹*)—, —N(R_{L}¹)—CO—O—, —O—, —S—, —SO₂—, —SO₂—N(R_{L}¹)—, —SO₂—O—, —CO—O—, —O—CO—, —O—CO—N(R_{L}¹)—, —N(R_{L}¹)— or —N(R_{L}¹)—SO₂—,

R_{A}¹² is hydrogen, a branched or unbranched, optionally substituted C₁-C₆-alkyl radical, a C₂-C₆-alkynyl or C₂-C₆-alkenyl radical which is optionally substituted by C₁-C₄-alkyl or aryl, or a 3- to 6-membered, saturated or unsaturated heterocycle which is substituted by up to three identical or different radicals, which can contain up to three different or identical heteroatoms O, N, S, a C₃-C₇-cycloalkyl, aryl or heteroaryl radical, where two radicals together can be a fused, saturated, unsaturated or aromatic carbocycle or heterocycle, which can contain up to three different or identical heteroatoms O, N, S, and the cycle can be optionally substituted or a further, optionally substituted, saturated, unsaturated or aromatic cycle can be fused to this cycle, or the radical R_{A}¹², together with R_{L}¹ or R_{L}¹*, forms a saturated or unsaturated C₃-C₇-heterocycle, which can optionally contain up to two further heteroatoms, selected from the group consisting of O, S or N,

R_{L}¹, R_{L}¹* independently of one another are hydrogen, a branched or unbranched, optionally substituted C₁-C₆-alkyl, C₁-C₆-alkoxyalkyl, C₂-C₆-alkenyl, C₂-C₁₂-alkynyl, CO—C₁-C₆-alkyl, CO—O—C₁-C₆-alkyl or SO₂—C₁-C₆-alkyl radical or an optionally substituted C₃-C₇-cycloalkyl, aryl, arylalkyl, CO—O-alkylenearyl, CO-alkylenearyl, CO-aryl, SO₂-aryl, hetaryl, CO-hetaryl- or SO₂-alkylenearyl radical,

R_{A}⁵ is a branched or unbranched, optionally substituted C₁-C₆-alkyl, arylalkyl, C₃-C₇-cycloalkyl or C₁-C₄-alkylene-C₃-C₇-cycloalkyl radical or an optionally substituted aryl, hetaryl, heterocycloalkyl or heterocycloalkenyl radical,

R_{A}⁶, R_{A}⁶ is hydrogen, a branched or unbranched, optionally substituted C₁-C₄-alkyl, —CO—O—C₁-C₄-alkyl, arylalkyl, —CO—O-alkylenearyl, —CO—O-allyl, —CO—C₁-C₄-alkyl, —CO-alkylenearyl, C₃-C₇-cycloalkyl or —CO-allyl radical or both radicals R_{A}⁶ and R_{A}⁶ in structural element I_{A}⁷ together are an optionally substituted, saturated, unsaturated or aromatic heterocycle, which in addition to the ring nitrogen can contain up to two further different or identical heteroatoms O, N, S,

R_{A}⁷ is hydrogen, —OH, —CN, —CONH₂, a branched or unbranched, optionally substituted C₁-C₄-alkyl, C₁-C₄-alkoxy, C₃-C₇-cycloalkyl or —O—CO—C₁-C₄-alkyl radical, or an optionally substituted arylalkyl, —O-alkylenearyl, —O—CO-aryl, —O—CO-alkylenearyl or —O—CO-allyl radical, or both radicals R_{A}⁶ and R_{A}⁷ together are an optionally substituted, unsaturated or aromatic heterocycle, which in addition to the ring nitrogen can contain up to two further different or identical heteroatoms O, N, S,

R_{A}⁸ is hydrogen, a branched or unbranched, optionally substituted C₁-C₄-alkyl, CO—C₁-C₄-alkyl, SO₂—C₁-C₄-alkyl or CO—O—C₁-C₄-alkyl radical or an optionally substituted aryl, CO-aryl, SO₂-aryl, CO—O-aryl, CO-alkylenearyl, SO₂-alkylenearyl, CO—O-alkylenearyl or alkylenearyl radical,

R_{A}⁹, R_{A}¹⁰ independently of one another are hydrogen, —CN, halogen, a branched or unbranched, optionally substituted C₁-C₆-alkyl radical or an optionally substituted aryl, arylalkyl, hetaryl, C₃-C₇-cycloalkyl radical or a radical CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}¹⁵R_{A}¹⁶, or both radicals R_{A}⁹ and R_{A}¹⁰ together in structural element I_{A}¹⁴ are a 5- to 7-membered saturated, unsaturated or aromatic carbocycle or heterocycle, which can contain up to three different or identical heteroatoms O, N, S and is optionally substituted by up to three identical or different radicals,

R_{A}¹¹ is hydrogen, —CN, halogen, a branched or unbranched, optionally substituted C₁-C₆-alkyl radical or an optionally substituted aryl, arylalkyl, hetaryl or C₃-C₇-cycloalkyl radical or a radical CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}¹⁵R_{A}¹⁶,

R_{A}¹⁷ is hydrogen or in structural element I_{A}¹⁶ both radicals R_{A}⁹ and R_{A}¹⁷ together are a 5- to 7-membered saturated, unsaturated or aromatic heterocycle, which in addition to the ring nitrogen can contain up to three different or identical heteroatoms O, N, S and is optionally substituted by up to three identical or different radicals,

R_{A}¹⁸, R_{A}¹⁹ independently of one another are hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- or bisalkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical, or a radical —SO₂—R₄, —CO—OR₄, —CO—NR₄R₄* or —CO—R₄,

Z₁, Z₂, Z₃, Z₄ independently of one another are nitrogen, C—H, C-halogen or a branched or unbranched, optionally substituted C—C₁-C₄-alkyl or C—C₁-C₄-alkoxy radical

Z₅ is NR_{A}⁸, oxygen or sulfur.

In a further, very particularly preferred embodiment, the structural element A is a structural element of the formula I_{A}¹, I_{A}⁴₁ I_{A}⁷, I_{A}⁸, I_{A}⁹ or I_{A}¹⁸.

A branched or unbranched, optionally substituted C₁-C₆-alkyl radical is understood as meaning for R_{A}¹ or R_{A}² independently of one another, for example, the corresponding radicals described above for R₁, preferably methyl or trifluoromethyl.

The branched or unbranched, optionally substituted radical CO—C₁-C₆-alkyl is composed for R_{A}¹ or R_{A}² in the structural elements I_{A}¹, I_{A}² I_{A}³ or I_{A}¹⁷, for example, of the group CO and the branched or unbranched, optionally substituted C₁-C₆-alkyl radicals described above for R_{A}¹ or R_{A}².

Optionally substituted hetaryl, hetarylalkyl, aryl, arylalkyl or C₃-C₇-cycloalkyl radicals are understood as meaning for R_{A}¹ or R_{A}² independently of one another, for example, the corresponding radicals described above for R₁.

The optionally substituted radicals CO—O—R_{A}¹⁴, O—R_{A}¹⁴, S—R_{A}¹⁴, NR_{A}¹⁵R_{A}¹⁶, CO—NR_{A}¹⁵R_{A}¹⁶ or SO₂NR_{A}¹⁵R_{A}¹⁶ are composed for R_{A}¹ or R_{A}², for example, of the groups CO—O, O, S, N, CO—N or SO₂—N and the radicals R_{A}¹⁴, R_{A}¹⁵ or R_{A}¹⁶ which are described below in greater detail.

R_{A}¹³ and R_{A}^{13*} are independently of one another, for example, fluorine, chlorine, bromine or iodine,

a branched or unbranched, optionally substituted C₁-C₆-alkyl radical, such as described above, for example, for R₁, preferably methyl or trifluoromethyl or an optionally substituted aryl, arylalkyl, hetaryl or C₃-C₇-cycloalkyl radical or a radical CO—O—R_{A}¹⁴ O—R_{A}¹⁴, S—R_{A}¹⁴, NR_{A}¹⁵R_{A}¹, SO₂NR_{A} R_{A} or CO—N R_{A}R_{A}such as in each case described above for R_{A}¹.

Preferred radicals for R_{A}¹³ and R_{A}^{13*} are the radicals hydrogen, F, Cl, a branched or unbranched, optionally substituted C₁-C₆-alkyl radical, optionally substituted aryl or arylalkyl or a radical CO—O—R_{A}O—R_{A}, NR_{A}¹⁵R_{A}¹, SO₂—NR_{A}¹⁵R_{A}¹ or CO—NR_{A}¹⁵R_{A}¹⁶.

A branched or unbranched, optionally substituted C₁-C₆-alkyl, C₃-C₇-cycloalkyl, alkylenecycloalkyl, alkylene-C₁-C₄-alkoxy, C₂-C₆-alkenyl or C₂-C₆-alkynyl radical is understood as meaning for R_{A}¹⁴ in structural element A, for example, the corresponding radicals described above for R₁.

Optionally substituted aryl, arylalkyl, hetaryl or alkylhetaryl radicals are understood as meaning for R_{A}¹⁴ in structural element A, for example, the corresponding radicals described above for R₁.

Preferred radicals for R_{A}¹⁴ are hydrogen, a branched or unbranched, optionally substituted C₁-C₆-alkyl radical and optionally substituted benzyl.

A branched or unbranched, optionally substituted C₁-C₆-alkyl or arylalkyl radical or an optionally substituted C₃-C₇-cycloalkyl, aryl, hetaryl or hetarylalkyl radical is understood as meaning for R_{A}¹⁵ or R_{A}¹⁶ independently of one another, for example, the corresponding radicals described above for R_{A}¹⁴.

The branched or unbranched, optionally substituted CO—C₁-C₆-alkyl, SO₂—C₁-C₆-alkyl, COO—C₁-C₆-alkyl, CO—NH—C₁-C₆-alkyl, COO-alkylenearyl, CO—NH-alkylenearyl, CO—NH-alkylenehetaryl or SO₂-alkylenearyl radicals or the optionally substituted CO-aryl, SO₂-aryl, CO—NH-aryl, CO—NH-hetaryl or CO-hetaryl radicals are composed for R_{A}¹⁵ or R_{A}¹⁶, for example, of the corresponding groups —CO—, —SO₂—, —CO—O—, —CO—NH— and the corresponding branched or unbranched, optionally substituted C₁-C₆-alkyl, hetarylalkyl or arylalkyl radicals described above or the corresponding optionally substituted aryl or hetaryl radicals.

A radical —(CH₂)ₙ—(X_{A})ⱼ—R_{A}¹² is understood as meaning for R_{A}³ or R_{A}⁴ independently of one another a radical which is composed of the corresponding radicals —(CH₂)ₙ—, (X_{A)}ⱼ and R_{A}¹². Here, n can be 0, 1, 2 or 3 and j 0 or 1.

X_{A} is a doubly bonded radical, selected from the group consisting of —CO—, —CO—N(R_{L}¹), —N(R_{L}¹)—CO—, —N(R_{L}¹)—CO—N(R_{L}¹*)—, —N(R_{L}¹)—CO—O—, —O—, —S—, —SO₂—, —SO₂—N(R_{L}¹)—, —SO₂—O—, —CO—O—, —O—CO—, —O—CO—N(R_{L}¹)—, —N(R_{L}¹)— or —N(R_{L}¹)—SO₂—.

R_{A}¹² is hydrogen,

a branched or unbranched, optionally substituted C₁-C₆-alkyl radical, such as described above for R₁,

a C₂-C₆-alkynyl or C₂-C₆-alkenyl radical which is optionally substituted by C₁-C₄-alkyl or aryl, such as described above, for example, for R_{X}⁹,

or a 3- to 6-membered, saturated or unsaturated heterocycle which is substituted by up to three identical or different radicals, which can contain up to three different or identical heteroatoms O, N, S, such as, for example, optionally substituted 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 2-(1,3,4-thiadiazolyl), 2-(1,3,4)-oxadiazolyl, 3-isoxazolyl, 4-isoxazolyl, 5-isoxazolyl, triazinyl.

Furthermore, R_{A}¹² and R_{L}¹ or R_{L}¹* can together form a saturated or unsaturated C₃-C₇-heterocycle, which optionally can contain up to two further heteroatoms, selected from the group consisting of O, S and N.

Preferably, the radical R_{A}¹² together with the radical R_{L}¹ or R_{L}¹* forms a cyclic amine as C₃-C₇-heterocycle, in the case where the radicals are bonded to the same nitrogen atom, such as, for example, N-pyrrolidinyl, N-piperidinyl, N-hexahydroazepinyl, N-morpholinyl or N-piperazinyl, wherein the case of heterocycles which carry free amine protons, such as, for example, N-piperazinyl, the free amine protons can be replaced by customary amine protective groups, such as, for example, methyl, benzyl, Boc (tert-butoxycarbonyl), Z (benzyloxycarbonyl), tosyl, —SO₂—C₁-C₄-alkyl, —SO₂-phenyl or —SO₂-benzyl.

A branched or unbranched, optionally substituted C₁-C₆-alkyl, C₁-C₆-alkoxyalkyl, C₂-C₆-alkenyl, C₂-C₁₂-alkynyl, CO—C₁-C₆-alkyl, CO—O—C₁-C₆-alkyl or SO₂—C₁-C₆-alkyl radical or an optionally substituted C₃-C₇-cycloalkyl, aryl, arylalkyl, CO—O-alkylenearyl, CO-alkylenearyl, CO-aryl, SO₂-aryl, hetaryl, CO-hetaryl or SO₂-alkylenearyl radical is understood as meaning for R_{L}¹ and R_{L}¹* independently of one another, for example, the radicals described above for R_{X}¹⁴.

Preferred radicals fur R_{L}¹ and R_{L}¹* are independently of one another hydrogen, methyl, cyclopropyl, allyl and propargyl.

R_{A}³ and R_{A}⁴ can furthermore together form a 3- to 8-membered, saturated, unsaturated or aromatic N heterocycle which can additionally contain two further, identical or different heteroatoms O, N, or S, where the cycle can optionally be substituted or a further, optionally substituted, saturated, unsaturated or aromatic cycle can be fused to this cycle,

R_{A}⁵ is a branched or unbranched, optionally substituted C₁-C₆-alkyl, arylalkyl, C₁-C₄-alkyl-C₃-C₇-cycloalkyl or C₃-C₇-cycloalkyl radical or an optionally substituted aryl, hetaryl, heterocycloalkyl or heterocycloalkenyl radical, such as described above, for example, for R₃, R₄ and R₅.

R_{A}⁶ and R_{A}⁸ are independently of one another hydrogen, a branched or unbranched, optionally substituted

C₁-C₄-alkyl radical, such as, for example, optionally substituted methyl, ethyl, propyl, 1-methylethyl, butyl, 1-methylpropyl, 2-methylpropyl or 1,1-dimethylethyl,

—CO—O—C₁-C₄-alkyl or —CO—C₁-C₄-alkyl radical such as, for example, composed of the group consisting of —CO—O— and —CO— and the C₁-C₄-alkyl radicals described above,

arylalkyl radical, such as described above for R₁,

—CO—O-alkylenearyl or —CO-alkylenearyl radical such as, for example, composed of the group consisting of —CO—O— and —CO— and the arylalkyl radicals described above,

—CO—O-allyl or —CO-allyl radical,

or C₃-C₇-cycloalkyl radical, such as, for example, described above for R₁.

R_{A}⁷ is hydrogen, —OH, —CN, —CONH₂, a branched or unbranched, optionally substituted C₁-C₄-alkyl radical, for example, such as described above for R_{A}⁶, C₁-C₄-alkoxy, arylalkyl or C₃-C₇-cycloalkyl radical, for example, such as described above for R_{X}¹⁴, a branched or unbranched, optionally substituted —O—CO—C₁-C₄-alkyl radical, which is composed of the group —O—CO— and, for example, of the abovementioned C₁-C₄-alkyl radicals or an optionally substituted —O-alkylenearyl, —O—CO-aryl, —O—CO-alkylenearyl or —O—CO-allyl radical which is composed of the groups —O— or —O—CO— and, for example, of the corresponding radicals described above for R₁.

Furthermore, both radicals R_{A}⁶ and R_{A}⁷ together can form an optionally substituted, unsaturated or aromatic heterocycle, which additionally to the ring nitrogen can contain up to two further different or identical heteroatoms O, N, S.

A branched or unbranched, optionally substituted C₁-C₄-alkyl radical or an optionally substituted aryl or arylalkyl radical is understood as meaning for R_{A}⁸ in structural element A, for example, the corresponding radicals described above for R_{A}¹⁵, where the radicals CO—C₁-C₄-alkyl, SO₂—C₁-C₄-alkyl, CO—O—C₁-C₄-alkyl, CO-aryl, SO₂-aryl, CO—O-aryl, CO-alkylenearyl, SO₂-alkylenearyl or CO—O-alkylenearyl are composed analogously to the other composed radicals from the group consisting of CO, SO₂ and COO and, for example, of the corresponding C₁-C₄-alkyl, aryl or the arylalkyl radicals described above for R_{A}¹⁵ and these radicals can optionally be substituted.

A branched or unbranched, optionally substituted C₁-C₆-alkyl radical or an optionally substituted aryl, arylalkyl, hetaryl or C₃-C₇-cycloalkyl radical is understood in each case as meaning for R_{A}⁹ or R_{A}¹⁰ independently of one another, for example, the corresponding radicals described above for R_{A}¹⁴, preferably methyl or trifluoromethyl.

A radical CO—O—R_{A}¹⁴, O—R_{A}¹⁴, S—R_{A}¹⁴, SO₂—NR_{A}¹⁵R_{A}¹⁶, NR_{A}¹⁵R_{A}¹⁶ or CO—NR_{A}¹⁵R_{A}¹⁶ is understood in each case as meaning for R_{A}⁹ or R_{A}¹⁰ independently of one another, for example, the corresponding radicals described above for R_{A}¹³.

Furthermore, both radicals R_{A}⁹ and R_{A}¹⁰ together in structural element I_{A}¹⁴ can form a 5- to 7-membered saturated, unsaturated or aromatic carbocycle or heterocycle, which can contain up to three different or identical heteroatoms O, N, S and is optionally substituted by up to three identical or different radicals.

Substituents are in this case in particular understood as meaning halogen, CN, a branched or unbranched, optionally substituted C₁-C₄-alkyl radical, such as, for example, methyl or trifluoromethyl or the radicals O—R_{A}, S—R_{A}, NR_{A} R_{A}, CO—NR_{A}¹⁵R_{A}¹⁶ or —((R_{A}⁸)HN)C═N—R_{A}⁷.

A branched or unbranched, optionally substituted C₁-C₆-alkyl radical or an optionally substituted aryl, arylalkyl, hetaryl, C₃-C₇-cycloalkyl radical or a radical CO—O—R_{A}¹⁴, O—R_{A}¹⁴, S—R_{A}, NR_{A}¹⁴R_{A}, SO₂—NR_{A}¹⁵R_{A}¹⁶ or CO—NR_{A}¹⁵R_{A}¹⁶ are understood as meaning for R_{A}¹¹ for example, the corresponding radicals described above for R_{A}⁹.

Furthermore, in structural element I_{A}¹⁶ both radicals R_{A}⁹ and R_{A}¹⁷ together form a 5- to 7-membered saturated, unsaturated or aromatic heterocycle, which additionally to the ring nitrogen can contain up to three different or identical heteroatoms O, N, S and is optionally substituted by up to three identical or different radicals

A branched or unbranched, optionally substituted C₁-C₈-alkyl, C₂-C₆-alkenyl, C₂-C₆-alkynyl, C₁-C₅-alkylene-C₁-C₄-alkoxy-, mono- and bisalkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical, or a radical —SO₂—R₃, —CO—OR₃, CO—NR₃R₃* or —CO—R₃ is understood as meaning for R_{A}¹⁸ and R_{A}¹⁹ independently of one another, for example, the radicals described above for R₄, preferably hydrogen or a branched or unbranched, optionally substituted C₁-C₈-alkyl radical.

Z¹, Z², Z³, Z⁴ are independently of one another nitrogen, C—H, C-halogen, such as, for example, C—F, C—Cl, C—Br or C—I or a branched or unbranched, optionally substituted C—C₁-C₄-alkyl radical which is composed of a carbon radical and, for example, a C₁-C₄-alkyl radical described above for R_{A}⁶ or a branched or unbranched, optionally substituted C—C₁-C₄-alkoxy radical which is composed of a carbon radical and, for example, a C₁-C₄-alkoxy radical described above for R_{A}⁷.

Z⁵ is oxygen, sulfur or a radical NR_{A}⁸.

Preferred structural elements A are composed of at least one preferred radical of the radicals belonging to the structural element A, while the remaining radicals are widely variable.

Particularly preferred structural elements A are composed of the preferred radicals of the structural element A.

In a further preferred embodiment, the spacer structural element E is a structural element of the formula I_{E}

(NR_{E}¹)ᵢ-E₁-(U_{E})_{g} (I_{E}),

where

(NR_{E}¹)ᵢ is the A-terminal end and (U_{E})_{g} the pyrimidinone-terminal end of the spacer structural element E,

U_{E} is oxygen, sulfur or NR_{E}², in particular NR_{E}²,

g is 0 or 1, in particular 1,

i is 0 or 1,

R_{E}¹ and R_{E}² independently of one another are hydrogen, a branched or unbranched, optionally substituted C₁-C₆-alkyl, alkoxyalkyl, alkylenearyl, alkylenealkynyl, hetaryl, CO—C₁-C₆-alkyl, CO—O—C₁-C₆-alkyl or SO₂—C₁-C₆-alkyl radical or an optionally substituted C₃-C₇-cycloalkyl, CO—O-alkylenearyl, CO-alkylenearyl, CO-aryl, SO₂-aryl, CO-hetaryl or SO₂-alkylenearyl radical, SO₂-hetaryl, SO₂-alkylenehetaryl,

particularly preferably hydrogen, a branched or unbranched, optionally substituted C₁-C₆-alkyl, alkylenearyl, alkylenealkynyl, hetaryl or an optionally substituted C₃-C₇-cycloalkyl radical,

in particular hydrogen, methyl, cyclopropyl, allyl or propargyl, and

E₁ is a structural element of the formula I_{E1}

—(CR_{E}³R_{E}⁴)ₖ₁-(L_{E})ₖ₂-(CR_{E}⁵R_{E}⁶)ₖ₃-(Q_{E})ₖ₄—(CR_{E}⁷R_{E}⁸)ₖ₅-(T_{E})ₖ₆—(CR_{E}⁹R_{E}¹⁰)ₖ₇— I_{E1}

where

k2, k4, k6 are 0 or 1,

k1,k3, k5, k7 are 0, 1 or 2,

R_{E}³, R_{E}⁴, R_{E}⁵, R_{E}⁶, R_{E}⁷, R_{E}⁸, R_{E}⁹, R_{E}¹⁰ independently of one another are hydrogen, halogen, a hydroxyl group, a branched or unbranched, optionally substituted C₁-C₆-alkyl, C₂-C₆-alkenyl, C₂-C₆-alkynyl or alkylenecycloalkyl radical, a radical —(CH₂)_{X}—(Y_{E})_{z}—R_{E}¹¹, an optionally substituted C₃-C₇-cycloalkyl, aryl, arylalkyl, hetaryl or hetarylalkyl radical or independently of one another in each case two radicals R_{E}³ and R_{E}⁴ or R_{E}⁵ and R_{E}⁶ or R_{E}⁷ and R_{E}⁸ or R_{E}⁹ and R_{E}¹⁰ together are a 3- to 7-membered, optionally substituted, saturated or unsaturated carbo- or heterocycle, which can contain up to three heteroatoms from the group consisting of O, N and S,

x is 0, 1, 2, 3 or 4,

z is 0 or 1,

Y_{E} is —CO—, —CO—N(R_{y}²)—, —N(R_{y}²)—CO—N(R_{y}²*)—, —N(R_{y}²)—CO—O—, —O—, —SO₂, —SO₂—, —SO₂—N(R_{y}²)—, —SO₂—O—, —CO—O—, —O—CO—, —O—CO—N(R_{y}²)—, —N(R_{y}²)— or —N(R_{y}²)—SO₂—,

R_{y}², R_{y}²* independently of one another are hydrogen, a branched or unbranched, optionally substituted C₁-C₆-alkyl, C₂-C₆-alkenyl, C₂-C₈-alkynyl, CO—C₁-C₆-alkyl, CO—O—C₁-C₆-alkyl or SO₂—C₁-C₆-alkyl radical or an optionally substituted hetaryl, hetarylalkyl, arylalkyl, C₃-C₇-cycloalkyl, CO—O-alkylenearyl, CO-alkylenearyl, CO-aryl, SO₂-aryl, CO-hetaryl or SO₂-alkylenearyl radical,

R_{E}¹¹ is hydrogen, a hydroxyl group, CN, halogen, a branched or unbranched, optionally substituted C₁-C₆-alkyl radical, an optionally substituted C₃-C₇-cycloalkyl, aryl, heteroaryl or arylalkyl radical, a C₂-C₆-alkynyl or C₂-C₆-alkenyl radical optionally substituted by C₁-C₄-alkyl or aryl, an optionally substituted C₆-C₁₂-bicycloalkyl, C₁-C₆-alkylene-C₆-C₁₂-bicycloalkyl, C₇-C₂₀-tricycloalkyl or C₁-C₆-alkylene-C₇-C₂₀-tricycloalkyl radical, or a 3- to 8-membered, saturated or unsaturated heterocycle substituted by up to three identical or different radicals, which can contain up to three different or identical heteroatoms O, N, S, where two radicals together can be a fused, saturated, unsaturated or aromatic carbocycle or heterocycle, which can contain up to three different or identical heteroatoms O, N, S, and the cycle can be optionally substituted or a further, optionally substituted, saturated, unsaturated or aromatic cycle can be fused to this cycle, or the radical R_{E}¹¹ together with R_{y}² or R_{y}²* forms a saturated or unsaturated C₃-C₇-heterocycle, which optionally can contain up to two further heteroatoms, selected from the group consisting of O, S and N,

L_{E}, T_{E} independently of one another are CO, CO—NR_{E}¹², NR_{E}¹²—CO, sulfur, SO, SO₂, SO₂—NR_{E}¹², NR_{E}¹²—SO₂, CS, CS—NR_{E}¹², NR_{E}¹²—CS, CS—O, O—CS, CO—O, O—CO, oxygen, ethynylene, CR_{E}¹³—O—CR_{E}¹⁴, C(═CR_{E}¹³R_{E}¹⁴), CR_{E}¹³═CR_{E}¹⁴, —CR_{E}¹³(OR_{E}¹⁵)—CHR_{E}¹⁴—, —CHR_{E}¹³—CR_{E}¹⁴ (OR_{E}¹⁵)—,

R_{E}¹² is hydrogen, a branched or unbranched, optionally substituted C₁-C₆-alkyl, C₂-C₆-alkenyl, C₂-C₈-alkynyl or an optionally substituted C₃-C₇-cycloalkyl, hetaryl, arylalkyl or hetarylalkyl radical or a radical CO—R_{E}¹⁶, COOR_{E}¹⁶ or SO₂—R_{E}¹⁶,

R_{E}¹³, R_{E}¹⁴ independently of one another are hydrogen, a hydroxyl group, a branched or unbranched, optionally substituted C₁-C₆-alkyl, C₁-C₄-alkoxy, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,

R_{E}¹⁵ is hydrogen, a branched or unbranched, optionally substituted C₁-C₆-alkyl, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,

R_{E}¹⁶ is hydrogen, a hydroxyl group, a branched or unbranched, optionally substituted C₁-C₆-alkyl, C₂-C₆-alkenyl, C₂-C₆-alkynyl or C₁-C₅-alkylene-C₁-C₄-alkoxy radical, or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-C₃-C₇-heterocycloalkyl, C₁-C₄-alkylene-C₃-C₇-heterocycloalkenyl or hetarylalkyl radical and

Q_{E} is an optionally substituted 4- to 11-membered mono- or polycyclic, aliphatic or aromatic hydrocarbon, which can contain up to 6 double bonds and up to 6 identical or different heteroatoms, selected from the group consisting of N, O and S, where the ring carbons or ring nitrogens can optionally be substituted.

U_{E} in structural element E is preferably sulfur or NR_{E}² and in particular NR_{E}².

The coefficients h and i are independently of one another 0 or 1.

In a preferred embodiment, the coefficient i is 1.

A branched or unbranched, optionally substituted C₁-C₆-alkyl, C₁-C₆-alkoxyalkyl, C₂-C₆-alkenyl, C₂-C₁₂-alkynyl or arylalkyl radical or an optionally substituted aryl, hetaryl or C₃-C₇-cycloalkyl is understood as meaning for R_{E}¹ and R_{E}² in structural element E independently of one another, for example, the corresponding radicals described above for R_{X}¹⁴.

The branched or unbranched, optionally substituted radicals CO—C₁-C₆-alkyl, CO—O—C₁-C₆-alkyl, CO—NH—C₁-C₆-alkoxyalkyl, CO—NH—C₁-C₆-alkyl or SO₂—C₁-C₆-alkyl radical or the optionally substituted radicals CO—O-alkylenearyl, CO—NH-alkylenearyl, CO-alkylenearyl, CO-aryl, CO—NH-aryl, SO₂-aryl, CO-hetaryl, SO₂-alkylenearyl, SO₂-hetaryl or SO₂-alkylenehetaryl are composed for R_{E}¹ and R_{E}² independently of one another, for example, of the corresponding groups CO, COO, CONH or SO₂ and the corresponding radicals mentioned above.

Preferred radicals for R_{E}¹ or R_{E}² are independently of one another hydrogen, a branched or unbranched, optionally substituted C₁-C₆-alkyl, C₁-C₆-alkoxy, C₂-C₆-alkenyl, C₂-C₁₂-alkynyl or arylalkyl radical, or an optionally substituted hetaryl or C₃-C₇-cycloalkyl radical.

Particularly preferred radicals für R_{E}¹ or R_{E}² are hydrogen, methyl, cyclopropyl, allyl or propargyl.

A branched or unbranched, optionally substituted C₁-C₆-alkyl, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical is understood as meaning for R_{E}³, R_{E}⁴, R_{E}⁵, R_{E}⁶, R_{E}⁷, R_{E}⁸, R_{E}⁹ or R_{E}¹⁰ independently of one another, for example, the corresponding radicals mentioned above for R_{X}¹.

The radical —(CH₂)_{X}—(Y_{E})_{z}—R_{E}¹¹ is composed of a CO—C₄-alkylene radical, a bond element Y_{E} preferably selected from the group consisting of —CO—N(R_{y}²)—, —N(R_{y}²)—CO—, —O—, —SO₂—N(R_{y}²)—, —N(R_{y}²)— or —N(R_{y}²)—SO₂—, and the radical R_{E}¹¹, where

R_{y}² and R_{y}²* preferably independently of one another are hydrogen, methyl, cyclopropyl, allyl, propargyl, and

R_{E}¹¹ for example, is a 3- to 8-membered, saturated or unsaturated heterocycle substituted by up to three identical or different radicals, which can contain up to three different or identical heteroatoms O, N, S, where two radicals together can be a fused, saturated, unsaturated or aromatic carbocycle or heterocycle, which can contain up to three different or identical heteroatoms O, N, S, and the cycle can be optionally substituted or a further, optionally substituted, saturated, unsaturated or aromatic cycle can be fused to this cycle, such as, for example, optionally substituted 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 2-(1,3,4-thiadiazolyl), 2-(1,3,4)-oxadiazolyl, 3-isoxazolyl, 4-isoxazolyl, 5-isoxazolyl or triazinyl.

Preferably, the radicals R_{E}¹¹ and R_{y}² or R_{y}²* together form a cyclic amine as C₃-C₇-heterocycle, in the case where the radicals are bonded to the same nitrogen atom, such as, for example, N-pyrrolidinyl, N-piperidinyl, N-hexahydroazepinyl, N-morpholinyl or N-piperazinyl, where in the case of heterocycles which carry free amine protons, such as, for example, N-piperazinyl, the free amine protons can be replaced by customary amine protective groups, such as, for example, methyl, benzyl, Boc (tert-butoxycarbonyl), Z (benzyloxycarbonyl), tosyl, —SO₂—C₁-C₄-alkyl, —SO₂-phenyl or —SO₂-benzyl.

Preferred radicals for R_{E}³, R_{E}⁴, R_{E}⁵, R_{E}⁶, R_{E}⁷, R_{E}⁸, R_{E}⁹ or R_{E}¹⁰ are independently of one another hydrogen, a branched or unbranched, optionally substituted C₁-C₆-alkyl radical, optionally substituted aryl or the radical —(CH₂)_{X}—(Y_{E})_{z}—R_{E}¹¹.

In a preferred embodiment of the structural element E₁, independently of one another one radical of R_{E}³ and R_{E}⁴ or R_{E}⁵ and R_{E}⁶ or R_{E}⁷ and R_{E}⁸ or R_{E}⁹ and R_{E}¹⁰ is hydrogen or methyl.

In a particularly preferred embodiment of the structural element E₁ the radicals R_{E}³, R_{E}⁴, R_{E}⁵, R_{E}⁶, R_{E}⁷, R_{E}⁸, R_{E}⁹ or R_{E}¹⁰ independently of one another are hydrogen or methyl.

L_{E} and T_{E} independently of one another are preferably CO—NR_{E}¹², NR_{E}¹²—CO, SO₂—NR_{E}¹², NR_{E}¹²—SO₂ or oxygen.

R_{E}¹² is preferably hydrogen, methyl, allyl, propargyl and cyclopropyl.

A branched or unbranched, optionally substituted C₀-C₆-alkyl, C₂-C₆-alkenyl or C₂-C₆-alkynyl radical or an optionally substituted C₃-C₇-cycloalkyl, aryl, arylalkyl, hetaryl or hetarylalkyl radical is understood as meaning for R_{E}¹³, R_{E}¹⁴ or R_{E}¹⁵ independently of one another, for example, the corresponding radicals described above for R_{X}¹.

A branched or unbranched, optionally substituted C₁-C₄-alkoxy radical is understood as meaning for R_{E}¹³ or R_{E}¹⁴ independently of one another, for example, the C₁-C₄-alkoxy radicals described above for R_{A}¹⁴.

Preferred alkylenecycloalkyl radicals are for R_{E}¹³, R_{E}¹⁴ or R_{E}¹⁵ independently of one another, for example, the C₁-C₄-alkylene-C₃-C₇-cycloalkyl radicals described above for R_{X}¹.

A branched or unbranched, optionally substituted C₁-C₆-alkyl, C₂-C₆-alkenyl, C₂-C₆-alkynyl or C₁-C₅-alkylene-C₁-C₄-alkoxy radical, or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-C₃-C₇-heterocycloalkyl, C₁-C₄-alkylene-C₃-C₇-heterocycloalkenyl or hetarylalkyl radical is understood as meaning for R_{E}¹⁶, for example, the corresponding radicals described above for R₄.

An optionally substituted 4- to 11-membered mono- or polycyclic aliphatic or aromatic hydrocarbon, which can contain up to 6 double bonds and up to 6 identical or different heteroatoms, selected from the group consisting of N, O, S, where the ring carbons or ring nitrogens can optionally be substituted is understood as meaning for Q_{E} preferably optionally substituted arylene, such as, for example, optionally substituted phenylene or naphthylene, optionally substituted hetarylene such as, for example, the radicals

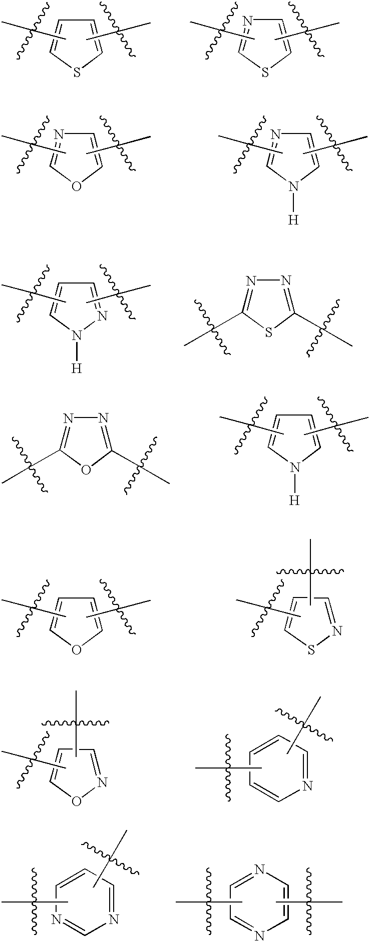

and their substituted or fused derivatives, or radicals of the formulae I_{E}¹ to I_{E}¹¹,

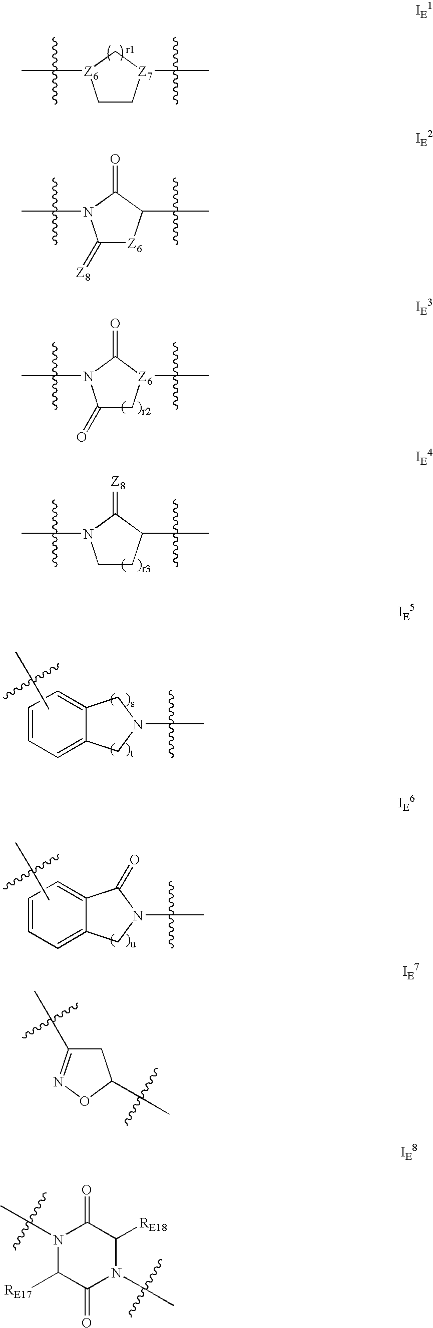

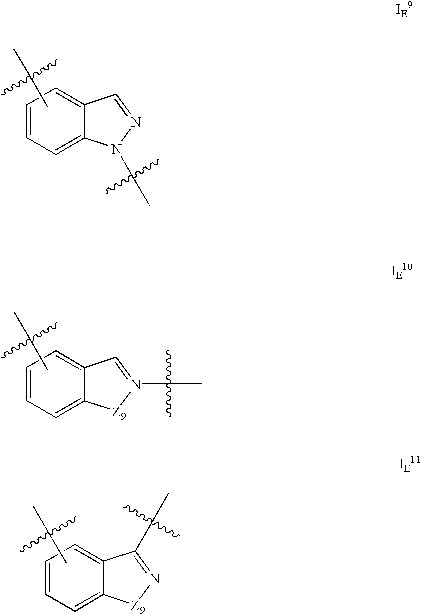

where the incorporation of the radicals can take place in both orientations.

Z⁶ and Z⁷ are independently of one another CH or nitrogen.

Z⁸ is oxygen, sulfur or NH

Z⁹ is oxygen, sulfur or NR_{E}¹⁹.

r1, r2, r3 and t are independently of one another 0, 1, 2 or 3.

s and u are independently of one another 0, 1 or 2.

Particularly preferably, Q_{E} is optionally substituted phenylene, a radical

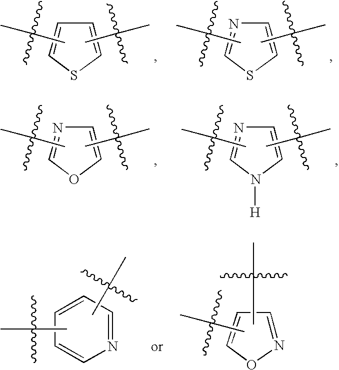

and their substituted or fused derivatives, or radicals of the formulae I_{E}¹, I_{E}², I_{E}³, I_{E}⁴ and I_{E}⁷, where the incorporation of the radicals can take place in both orientations. Optionally substituted phenylene or a radical of the formula I_{E}¹ are particularly preferred.

R_{E}¹⁷ and R_{E}¹⁸ are independently of one another hydrogen, —NO₂, —NH₂, —CN, —COOH, a hydroxyl group, halogen, a branched or unbranched, optionally substituted C₁-C₆-alkyl, C₁-C₄-alkoxy, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical, such as in each case described above.

R_{E}¹⁹ is independently of one another hydrogen, a branched or unbranched, optionally substituted C₁-C₆-alkyl, C₁-C₆-alkoxyalkyl, C₃-C₁₂-alkynyl, CO—C₁-C₆-alkyl, CO—O—C₁-C₆-alkyl- or SO₂—C₁-C₆-alkyl radical or an optionally substituted C₃-C₇-cycloalkyl, aryl, arylalkyl, CO—O-alkylenearyl, CO-alkylenearyl, CO-aryl, SO₂-aryl, hetaryl, CO-hetaryl or SO₂-alkylenearyl radical, preferably hydrogen or a branched or unbranched, optionally substituted C₁-C₆-alkyl radical.

Preferred structural elements E are composed of at least one preferred radical of the radicals belonging to the structural element E, while the remaining radicals are widely variable.

Particularly preferred structural elements E are composed of the preferred radicals of the structural element E.

A radical hydrolyzable to COOH is understood as meaning a radical which changes into a group COOH after hydrolysis.

An example of a radical T hydrolyzable to COOH which may be mentioned is the group

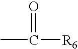

in which R₆ has the following meaning:

a) OM, where M can be a metal cation, such as an alkali metal cation, such as lithium, sodium, potassium, the equivalent of an alkaline earth metal cation, such as calcium, magnesium and barium or an environmentally compatible organic ammonium ion such as, for example, primary, secondary, tertiary or quaternary C₁-C₄-alkylammonium or ammonium ion, such as, for example, ONa, OK or OLi,

b) a branched or unbranched, C₁-C₈-alkoxy radical optionally substituted by halogen, such as, for example, methoxy, ethoxy, propoxy, 1-methylethoxy, butoxy, 1-methylpropoxy, 2-methylpropoxy, 1,1-dimethylethoxy, in particular methoxy, ethoxy, 1-methylethoxy, pentoxy, hexoxy, heptoxy, octoxy, difluoromethoxy, trifluoromethoxy, chlorodifluoromethoxy, 11-fluoroethoxy, 2-fluoroethoxy, 2,2-difluoroethoxy, 1,1,2,2-tetrafluoroethoxy, 2,2,2-trifluoroethoxy, 2-chloro-1,1,2-trifluoroethoxy or pentafluoroethoxy,

c) a branched or unbranched C₁-C₄-alkylthio radical optionally substituted by halogen, such as methylthio, ethylthio, propylthio, 1-methylethylthio, butylthio, 1-methylpropylthio, 2-methylpropylthio or 1,1-dimethylethylthio radical

d) an optionally substituted —O-alkylenearyl radical, such as, for example, —O-benzyl

e) R¹ is furthermore a radical —(O)ₘ, —N(R₇)(R₈), in which ml is 0 or 1 and R₇ and R₈, which can be identical or different, have the following meaning:

hydrogen,

a branched or unbranched, optionally substituted C₁-C₆-alkyl radical, C₂-C₆-alkenyl radical, C₂-C₆-alkynyl radical, C₃-C₈-cycloalkyl, or a phenyl radical, optionally mono- or polysubstituted, for example, mono- to trisubstituted, by halogen, nitro, cyano, C₀-C₄-alkyl, C₁-C₄-haloalkyl, C₁-C₄-alkoxy, C₁-C₄-haloalkoxy or C₁-C₄-alkylthio such as, for example, 2-fluorophenyl, 3-chlorophenyl, 4-bromophenyl, 2-methylphenyl, 3-nitrophenyl, 4-cyanophenyl, 2-trifluoro-methylphenyl, 3-methoxyphenyl, 4-trifluoroethoxyphenyl, 2-methylthiophenyl, 2,4-dichlorophenyl, 2-methoxy-3-methylphenyl, 2,4-dimethoxyphenyl, 2-nitro-5-cyanophenyl, 2,6-difluorophenyl,

or R₇ and R₈ together form an optionally substituted C₄-C₇-alkylene chain, e.g. substituted by C₁-C₄-alkyl, which is closed to give a cycle, which can contain a heteroatom, selected from the group consisting of oxygen, sulfur and nitrogen, such as, for example, —(CH₂)₄—, —(CH₂)₅—, —(CH₂)₆—, —(CH₂)₇—, —(CH₂)₂—O—(CH₂)₂—, —CH₂—S—(CH₂)₃—, —(CH₂)₂—O—(CH₂)₃—, —N H—(CH₂)₃—, —CH₂—NH—(CH₂)₂—, —CH₂—CH═CH—CH₂—, —CH═CH—(CH₂)₃—, —CO—(CH₂)₂—CO— or —CO—(CH₂)₃—CO—.

Preferred radicals T are —COOH, —CO—O—C₁-C₈-alkyl or —CO—O-benzyl.

In a further preferred embodiment, the sum of a, b, c and d in the spacer structural element X is less than 5. In particular, a, b, c, d are 0 or 1. Furthermore, the sum of a and b is preferably 1 and the sum of c and d preferably 0 or 1. Furthermore, e and/or f are preferably 0.

Furthermore, w is preferably 0 or 1, in particular 0.

Furthermore, in a preferred embodiment of X the radicals R_{X}¹—R_{X}⁸ independently of one another are hydrogen or methyl and the coefficients e and f are 0 or 1. Particularly preferably in this embodiment, w in W_{X} is 0.

R_{w}¹ is preferably hydrogen, a branched or unbranched, optionally substituted C₁-C₆-alkyl, alkylenearyl, alkylenealkynyl, hetaryl or C₃-C₇-cycloalkyl radical. In particular, R_{w}¹ is a hydrogen, methyl, cyclopropyl, allyl or propargyl radical.

The term C₁-C₆-alkyl radical is understood in the present invention as meaning, for example, optionally substituted methyl, ethyl, propyl, 1-methylethyl, butyl, 1-methyl-propyl, 2-methylpropyl, 1,1-dimethylethyl, pentyl, 1-methylbutyl, 2-methylbutyl, 1,2-dimethylpropyl, 1,1-dimethylpropyl, 2,2-dimethylpropyl, 1-ethylpropyl, hexyl, 1-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl or 1-ethyl-2-methylpropyl.

The term C₂-C₆-alkenyl radical in the present invention comprises, for example, optionally substituted vinyl, 2-propenyl, 2-butenyl, 3-butenyl, 1-methyl-2-propenyl, 2-methyl-2-propenyl, 2-pentenyl, 3-pentenyl, 4-pentenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-2-propenyl, 1-ethyl-2-propenyl, 2-hexenyl, 3-hexenyl, 4-hexenyl, 5-hexenyl, 1-methyl-2-pentenyl, 2-methyl-2-pentenyl, 3-methyl-2-pentenyl, 4-methyl-2-pentenyl, 3-methyl-3-pentenyl, 4-methyl-3-pentenyl, 1-methyl-4-pentenyl, 2-methyl-4-pentenyl, 3-methyl-4-entenyl, 4-methyl-4-pentenyl, 1,1-dimethyl-2-butenyl, 1,1-dimethyl-3-butenyl, 1,2-dimethyl-2-butenyl, 1,2-dimethyl-3-butenyl, 1,3-dimethyl-2-butenyl, 1,3-dimethyl-3-butenyl, 2,2-dimethyl-3-butenyl, 2,3-dimethyl-2-butenyl, 2,3-dimethyl-3-butenyl, 1-ethyl-2-butenyl, 1-ethyl-3-butenyl, 2-ethyl-2-butenyl, 2-ethyl-3-butenyl, 1,1,2-trimethyl-2-propenyl, 1-ethyl-1-methyl-2-propenyl or 1-ethyl-2-methyl-2-propenyl.

The term C₂-C₆-alkynyl radical in the present invention comprises, for example, optionally substituted ethynyl, 2-propynyl, 2-butynyl, 3-butynyl, 1-methyl-2-propynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 1-methyl-2-pentynyl, 1-methyl-2-pentynyl, 1-methyl-3-pentynyl, 1-methyl-4-pentynyl, 2-methyl-3-pentynyl, 2-methyl-4-pentynyl, 3-methyl-4-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 1-ethyl-2-butynyl, 1-ethyl-3-butynyl, 2-ethyl-3-butynyl or 1-ethyl-1-methyl-2-propynyl.

The term C₃-C₇-heterocycloalkyl radical comprises, for example, optionally substituted aziridinyl, diaziridinyl, oxiranyl, oxaziridinyl, oxetanyl, thiiranyl, thietanyl, pyrrolidinyl, piperazinyl, morpholinyl, piperidinyl, tetrahydrofuranyl, tetrahydropyranyl, 1,4-dioxanyl, hexahydroazepinyl, oxepanyl, 1,2-oxathiolanyl or oxazolidinyl.

The term C₃-C₇-heterocycloalkenyl radical comprises, for example, optionally substituted azirinyl, diazirinyl, thiirenyl, thietyl, pyrrolinyls, oxazolinyls, azepinyl, oxepinyl, α-pyranyl, β-pyranyl, γ-pyranyl, dihydropyranyls, 2,5-dihydropyrrolinyl or 4,5-dihydrooxazolyl.

The term C₃-C₇-cycloalkyl radical used above is to be understood as meaning, for example, optionally substituted cyclopropyl, cyclobutyl, cyclopentyl, cyclohexyl or cycloheptyl.

The term aryl radical is preferably to be understood as meaning optionally substituted phenyl, 1-naphthyl or 2-naphthyl.

The term arylalkyl radical preferably comprises optionally substituted benzyl or ethylenephenyl (homobenzyl).

The term hetaryl radical is preferably to be understood as meaning optionally substituted 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 3-isoxazolyl, 4-isoxazolyl, 5-isoxazolyl, thiadiazolyl, oxadiazolyl or triazinyl or their fused derivatives such as, for example, indazolyi, indolyl, benzothiophenyl, benzofuranyl, indolinyl, benzimidazolyl, benzothiazolyl, benzoxazolyl, quinolinyl or isoquinolinyl.

The term hetarylalkyl radical preferably comprises optionally substituted —CH₂-2-pyridyl, —CH₂-3-pyridyl, —CH₂-4-pyridyl, —CH₂-2-thienyl, —CH₂-3-thienyl, —CH₂-2-thiazolyl, —CH₂₄-thiazolyl, CH₂-5-thiazolyl, —CH₂—CH₂-2-pyridyl, —CH₂—CH₂-3-pyridyl, —CH₂—CH₂-4-pyridyl, —CH₂—CH₂-2-thienyl, —CH₂—CH₂-3-thienyl, —CH₂—CH₂-2-thiazolyl, —CH₂—CH₂-4-thiazolyl or —CH₂—CH₂-5-thiazolyl.

The term C₃-C₇-heterocycloalkenyl radical comprises, for example, optionally substituted azirinyl, diazirinyl, thiirenyl, thietyl, pyrrolinyls, oxazolinyls, azepinyl, oxepinyl, α-pyranyl, β-pyranyl, γ-pyranyl, dihydropyranyls, 2,5-dihydropyrrolinyl or 4,5-dihydrooxazolyl.

A halogen radical is understood as meaning for all radicals and substituents of the present invention, if not mentioned otherwise, for example, F, Cl, Br or I.

Optionally substituted radicals are understood as meaning the corresponding unsubstituted and substituted radicals. For all substituted radicals of the present invention, if the substituents are not specified in greater detail, suitable substituents independently of one another are up to 5 substituents, for example, selected from the following group:

—NO₂, —NH₂, —OH, —CN, —COOH, —O—CH₂—COOH, halogen, a branched or unbranched,

optionally substituted C₁-C₄-alkyl radical,

such as, for example, methyl, CF₃, C₂F₅ or CH₂F,

a branched or unbranched, optionally substituted —CO—O—C₁-C₄-alkyl, C₃-C₇-cycloalkyl,

C₁-C₄-alkoxy, C₁-C₄-thioalkyl, —N H—CO—O—C₁-C₄-alkyl, —O—CH₂—COO—C₁-C₄-alkyl, —NH—CO—C₁-C₄-alkyl, —CO—N H—C₁-C₄-alkyl, —N H—SO₂—C₁-C₄-alkyl, —SO₂—N H—C₁-C₄-alkyl, —N(C₁-C₄-alkyl)₂, —N H—C₁-C₄-alkyl, or —SO₂—C₁-C₄-alkyl radical, such as, for example, —SO₂—CF₃,

an optionally substituted —N H—CO-aryl, —CO—NH-aryl, —N H—CO—O-aryl, —N H—CO—O-alkylenearyl, —NH—SO₂-aryl, —SO₂—NH-aryl, —CO—NH-benzyl, —NH—SO₂-benzyl- or —SO₂—NH-benzyl radical, an optionally substituted radical —SO₂—NR₉R₁₀ or —CO—NR₉R₁₀ where the radicals R₉ and R₁₀ independently of one another can have the meaning such as R_{X}¹⁴ above or both radicals R₉ and R₁₀ together are a 3- to 6-membered, optionally substituted, saturated, unsaturated or aromatic heterocycle, which additionally to the ring nitrogen can contain up to three further different or identical heteroatoms O, N, S, and optionally two substituted radicals on this heterocycle together are a fused, saturated, unsaturated or aromatic carbocycle or heterocycle, which can contain up to three different or identical heteroatoms O, N, S and the cycle can be optionally substituted or a further, optionally substituted cycle can be a fused to this cycle.

In all terminally bonded, substituted hetaryl and hetarylalkyl radicals of the present invention, in addition to the above list of substituents, two substituents of the hetaryl moiety form a fused 5- to 7-membered, unsaturated or aromatic carbocycle.

The compounds of the formula I and also the intermediates for their preparation can have one or more asymmetrical substituted carbon atoms. The compounds can be present as pure enantiomers or pure diastereomers or as a mixture thereof. The use of an enantiomerically pure compound as active compound is preferred.

The compounds of the formula I can also be present in other tautomeric forms.

The compounds of the formula I can also be present in the form of physiologically tolerable salts.

The compounds of the formula I can also be present as prodrugs in a form in which the compounds of the formula I are released under physiological conditions. An example which may be referred to here is the group T, which in some cases contains groups which are hydrolyzable under physiological conditions to the free carboxylic acid group. Derivatized structural elements A and E are also suitable which release the structural element A or E under physiological conditions.

In preferred compounds of the formula I, in each case one of the three structural elements A-E-, pyrimidinone or X-T has the preferred range, while the remaining structural elements are widely variable.

In particularly preferred compounds of the formula 1, in each case two of the three structural elements A-E-, pyrimidinone or X-T have the preferred range, while the remaining structural elements are widely variable.

In very particularly preferred compounds of the formula 1, in each case all three structural elements A-E-, pyrimidinone or X-T have the preferred range, while the remaining structural element is widely variable.

Preferred compounds of the formula I have, for example, the preferred structural element X-T, while the structural elements A-E and pyrimidinone are widely variable.

Particularly preferred compounds of the formula I have, for example, the preferred structural element X-T and the preferred structural element pyrimidinone, while the structural elements E and A are widely variable.

Further particularly preferred compounds have the preferred structural elements E, pyrimidinone and X-T, while the structural element A is widely variable.

Further very particularly preferred compounds have the preferred structural elements A, pyrimidinone and X-T, while the structural element E is widely variable.

Further very particularly preferred compounds have the preferred structural elements A, E, pyrimidinone and X-T.

Particularly preferred compounds of the general formula A-E-pyrimidinone-X-T are listed below:

In the following list, the following abbreviations are used for the units A-E and pyrimidinone-X-T.

| A—E = | Abbreviation | A—E = | Abbreviation |
|---|---|---|---|
| 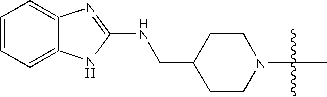 | Ambe | 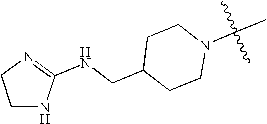 | Amdhim |
| 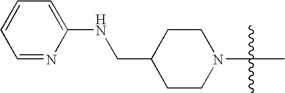 | Ampy | 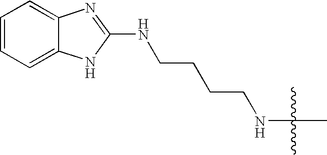 | Ambebu |
| 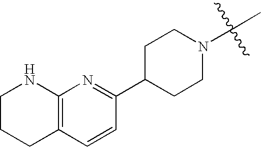 | Napht | 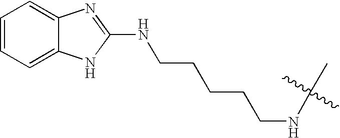 | Ambepe |
| 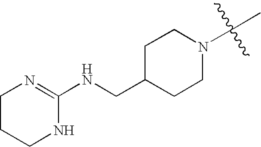 | Amdin | 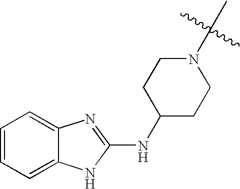 | Ambepi |
| Pyrimidinone-X—T = | Abbreviation | Pyrimidinone-X—T = | Abbreviation |
| 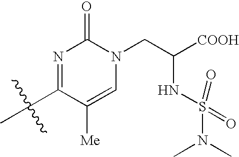 | Mepyr | 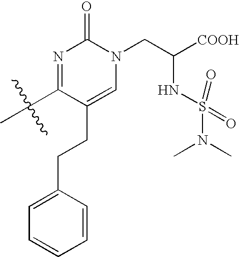 | Phenpyr |

1-Napht-Mepyr

2-Amdin-phenpyr

3-Amdhim-phenpyr

4-Ambepi-Mepyr

5-Napht-Phenpyr

6-Amdin-Mepyr

7-Ambepi-Phenpyr

8-Amdhim-Mepyr

9-Ampy-Mepyr

10-Ambe-Mepyr

11-Ambe-Phenpyr

12-Ambebu-Mepyr

13-Ambebu-Phenpyr

14-Ambepe-Mepyr

15-Ambepe-Phenpyr

16-Ampy-Phenpyr

Generally, the compounds of the general formula (I) and the starting substances used for their preparation can be prepared according to methods of organic chemistry known to the person skilled in the art, such as are described in standard works such as, for example, Houben-Weyl, “Methoden der Organischen Chemie”, Thieme-Verlag, Stuttgart, or March “Advanced Organic Chemistry”, 4th Edition, Wiley & Sons. Further preparation methods are also described in R. Larock, “Comprehensive Organic Transformations”, Weinheim 1989, in particular the preparation of alkenes, alkynes, halides, amines, ethers, alcohols, phenols, aldehydes, ketones, nitrites, carboxylic acids, esters, amides and acid chlorides.

The synthesis of compounds of the formula (I) can be carried out either according to the “classical” method in solution or on a polymeric support, in each case reaction conditions being used such as are known and suitable for the respective reactions. In this case, use can also be made of variants which are known per se, but not mentioned here.

The general synthesis of compounds of type I is described in schemes 1 and 2 below. If not stated otherwise, all starting materials and reagents are commercially available, or can be prepared from commercially obtainable precursors according to customary methods.

A general method for the preparation of compounds of the general formula I is described in WO 00/61551, pp. 215-225. This comprises the synthesis of the parent structure as well as the preparation of appropriate base units and spacer fragments.

The synthesis is carried out starting from appropriately substituted 4-thioxo-3,4-dihydropyrimidin-2(1H)-ones of the general formula (II) as intermediates. 4-Thioxo-3,4-dihydropyrimidin-2(1H)-ones of type (II) are known and can be prepared by known methods, such as are described, for example, in Katritzky and Rees, “Comprehensive Heterocyclic Chemistry” Pergamon Press, volume 3; pp. 135-139 and the literature cited there. A preferred method for the synthesis of 4-thioxo-3,4-dihydropyrimidin-2(1H)-ones consists, for example, in the addition of enamines to isothiocyanates with subsequent cyclization, as described by Goerdeler et al. in Chem. Ber. 1963, pp. 526-533, and Chem. Ber. 1965, pp. 1531-1542. Particularly preferably, 4-thioxo-3,4-dihydropyrimidin-2(1H)-ones can be prepared according to the method described by Lamon in J. Heterocycl. Chem. 1968, 5, 837-844, which is based on the reaction of an enamine with alkoxy- or aryloxycarbonyl isothiocyanate.

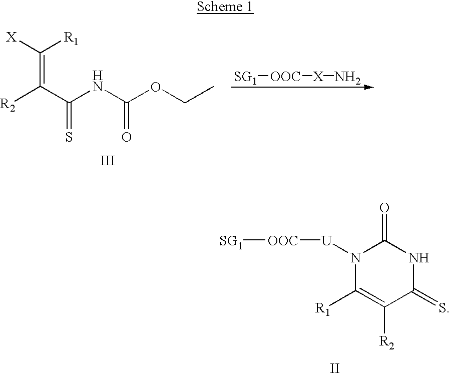

For the synthesis of compounds of the formula (I), appropriate enamine derivatives of the general formula (III), in which X is preferably a morpholine, pyrrolidine or piperidine radical, are reacted with primary amines with formation of the subst. 4-thioxo-3,4-dihydropyrimidin-2(1H)-ones (II) (Scheme 1). Carrying out the synthesis on a solid phase is particularly efficient, in that the carboxylic acid function is used as an anchor group for the linkage to a solid support (SG=solid support). Methods for synthesis on a solid phase are described in detail, for example, by Bunin in “The Combinatorial Index” (Academic Press, 1998).

For further reaction, the 4-thioxo group in compounds of the general formula 11 is alkylated according to standard methods with addition of a base.

The 4-thioxo group can then preferably be converted into the corresponding thiocyanate by alkylation with cyanogen bromide, as described, for example, in Tetrahedron Letters 1991, 32 (22), 2505-2508 (Scheme II). The thiocyanate can then be reacted with suitable amines, thiols or alcohols of the general formula A-E₁-(U_{E})_{g}—H according to

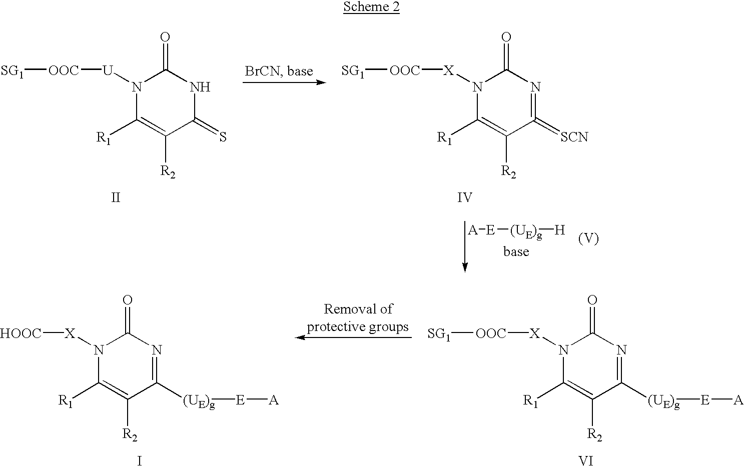

methods known to the person skilled in the art, possibly with addition of a base, to give the compounds of the general formula (IV) (Scheme 2). Removal of the protective group SG1 according to standard conditions (see below) leads to the compounds of the general formula (I). If SG1 is C₁₋₄-alkyl or benzyl, the compounds of the general formula (VI) correspond directly to the compounds of type I. The sulfamoyl radical contained in the fragment X can either be contained directly during the synthesis of IV in the component H₂N-T-COOSG₁, or introduced afterwards according to standard methods after removal of a suitable amino protective group; both variants are described in examples in the experimental section.

The protective groups SG used can be all protective groups which are known from peptide synthesis to the person skilled in the art and customary, as are also described in the standard works such as, for example, Bodanszky “The Practice of Peptide Synthesis”, 2nd Edition, Springer-Verlag 1994, and Bodanszky “Principles of Peptide Synthesis”, Springer-Verlag 1984. The removal of the protective groups in the compounds of the formula (VI) or the protective groups used in the preparation of the compounds (V) and (VI I) is likewise carried out under conditions such as are known to the person skilled in the art and described, for example, by Greene and Wuts in “Protective Groups in Organic Synthesis”, 2nd Edition, Wiley & Sons, 1991.

As amino protective groups Boc, Fmoc, benzyloxycarbonyl (Z), acetyl, Mtr are preferably used; as acid protective groups, such as, for example, SG₁, preferably C₁₋₄-alkyl such as, for example, methyl, ethyl, tert-butyl, or alternatively benzyl or trityl, or alternatively polymer-bound protective groups in the form of the commercially available polystyrene resins such as, for example, 2-chlorotrityl chloride resin or Wang resin (Bachem, Novabiochem).

The removal of acid-labile protective groups (e.g. Boc, tert-butyl, Mtr, trityl) can be carried out—depending on the protective group used—using organic acids such as trifluoroacetic acid (TFA), trichloracetic acid, perchloric acid, trifluoroethanol; but also inorganic acids such as hydrochloric acid or sulfuric acid, sulfonic acids such as benzene- or p-toluenesulfonic acid, the acids generally being employed in an excess. In the case of trityl, the addition of thiols such as, for example, thioanisole or thiophenol can be advantageous. The presence of an additional inert solvent is possible, but not always necessary. Suitable inert solvents are preferably organic solvents, for example, carboxylic acids such as acetic acid; ethers such as THF or dioxane; amides such as DMF or dimethylacetamide; halogenated hydrocarbons such as dichloromethane; alcohols such as methanol, isopropanol; or water. Mixtures of the solvents mentioned are also suitable. The reaction temperature for these reactions is between 10° C. and 50° C., they are preferably carried out in a range between 0° C. and 30° C.

Base-labile protective groups such as Fmoc are cleaved by treatment with organic amines such as dimethylamine, diethylamine, morpholine, piperidine as 5-50% solutions in CH₂Cl₂ or DMF. The reaction temperature for these reactions is between 10° C. and 50° C., they are preferably carried out in a range between 0° C. and 30° C.

Acid protective groups such as methyl or ethyl are preferably cleaved by basic hydrolysis in an inert solvent. The bases used are preferably alkali metal or alkaline earth metal hydroxides, preferably NaOH, KOH or LiOH; solvents used are all customary inert solvents such as, for example, hydrocarbons such as hexane, heptane, petroleum ether, toluene, benzene or xylene; chlorinated hydrocarbons such as trichloroethylene, 1,2-dichloroethane, carbon tetrachloride, chloroform, dichloromethane; alcohols such as methanol, ethanol, isopropanol, n-propanol, n-butanol or tert-butanol; ethers such as diethyl ether, methyl tert-butyl ether, diisopropyl ether, tetrahydrofuran, dioxane; glycol ethers such as ethylene glycol monomethyl ether or monoethyl ether, ethylene glycol dimethyl ether; ketones such as acetone, butanone; amides such as dimethylformamide (DMF), dimethylacetamide or acetamide; nitriles such as acetonitrile; sulfoxides such as dimethyl sulfoxide, sulfolane; N-methylpyrrolidone, 1,3-dimethyltetrahydro-2(1H)-pyrimidinone (DMPU), 1,3-dimethyl-2-imidazolidinone; nitro compounds such as nitromethane or nitrobenzene; water or mixtures of the solvents mentioned. The addition of a phase-transfer catalyst can be advantageous—depending on the solvent or solvent mixture used. The reaction temperature for these reactions is generally between −10° C. and 100° C.

Hydrogenolytically removable protective groups such as benzyloxycarbonyl (Z) or benzyl can be removed, for example, by hydrogenolysis in the presence of a catalyst (e.g. of a noble metal catalyst on active carbon as a support). Suitable solvents are those indicated above, in particular alcohols such as methanol, ethanol; amides such as DMF or dimethylacetamide; esters such as ethyl acetate. As a rule, the hydrogenolysis is carried out at a pressure of 1-200 bar and temperatures between 0° and 100° C.; the addition of an acid such as, for example, acetic acid or hydrochloric acid can be advantageous. The catalyst used is preferably 5-10% Pd on active carbon.

The synthesis of components of type E and A is generally carried out according to methods known to the person skilled in the art; the components used are either commercially available or accessible according to methods known from the literature. The synthesis of some of these components is described by way of example in the experimental section. General methods for the preparation of components of type E and A are described in WO 00/61551; the preparation of fragments of type I_{A}¹⁸ can be carried out analogously to WO 00/09503.

The object of the invention is furthermore achieved by a pharmaceutical preparation comprising at least one compound of the general formula (I) and customary excipients and/or vehicles.

The compounds according to the invention can be used for the treatment of diseases in which the interaction between integrins and their natural ligands is excessive or reduced.

Furthermore, the object is achieved by a process for the treatment and/or prophylaxis of diseases in which the interaction between the integrins and their natural ligands is excessive or reduced, by administering an efficacious amount of at least one compound of the general formula (I).

The pharmaceutical preparation according to the invention is described in more detail below.

The compounds according to invention can be administered orally or parenterally (subcutaneously, intravenously, intramuscularly, intraperitoneally) in a customary manner. Administration can also be carried out through the nasopharynx using vapours or sprays. Furthermore, the compounds according to the invention can be introduced by direct contact with the tissue concerned.

The dose depends on the age, condition and weight of the patient and on the manner of administration. As a rule, the daily dose of active compound is between approximately 0.5 and 50 mg/kg of body weight in the case of oral administration and between approximately 0.1 and 10 mg/kg of body weight in the case of parenteral administration.

The novel compounds can be administered in solid or liquid form in the customary pharmaceutical administration forms, e.g. as tablets, film-coated tablets, capsules, powders, granules, coated tablets, suppositories, solutions, ointments, creams or sprays. These are prepared in the customary manner. The active compounds can in this case be processed with the customary pharmaceutical excipients such as tablet binders, fillers, preservatives, tablet disintegrants, flow regulators, plasticizers, wetting agents, dispersants, emulsifiers, solvents, release-delaying agents, antioxidants and/or propellents (cf. H. Sucker et al.: Pharmazeutische Technologie [Pharmaceutical Technology], Thieme-Verlag, Stuttgart, 1991). The administration forms thus obtained normally contain the active compound in an amount of from 0.1 to 90% by weight.

The invention further relates to the compounds of the formula I for use as medicaments and the use of the compounds of the formula I for the production of medicaments for the treatment of diseases. The compounds of the formula I can be used for the treatment of human and animal diseases. The compounds of the formula I bind to integrin receptors. They are therefore preferably suitable as integrin-receptor ligands and for the production of medicaments for the treatment of diseases in which an integrin receptor is involved, in particular for the treatment of diseases in which the interaction between integrins and their natural ligands is dysregulated, i.e. is excessive or reduced.

Integrin receptor ligands are understood as meaning agonists and antagonists.

An excessive or reduced interaction is understood as meaning either an excessive or reduced expression of the natural ligand and/or of the integrin receptor and thus an excessive or reduced amount of natural ligand and/or integrin receptor or an increased or reduced affinity of the natural ligand for the integrin receptor.

The interaction between integrins and their natural ligands is dysregulated compared with the normal state, i.e. excessive or reduced, if this dysregulation does not correspond to the physiological state. An increased or reduced interaction can lead to pathophysiological situations.

The level of the dysregulation which leads to a pathophysiological situation is dependent on the individual organism and on the site and the nature of the disease.

Preferred integrin receptors for which the compounds of the formula I according to the invention can be used are the α₅β₁-, α₄β₁—, αᵥβ₅— and αᵥβ₃-integrin receptors.

Particularly preferably, the compounds of the formula I bind to the αᵥβ₃-integrin receptor and can thus particularly preferably be used as ligands of the αᵥβ₃-integrin receptor and for the treatment of diseases in which the interaction between αᵥβ₃-integrin receptor and its natural ligands is excessive or reduced.

The compounds of the formula I are preferably used for the treatment of the following diseases or for the production of medicaments for the treatment of the following diseases:

cardiovascular diseases such as atherosclerosis, restenosis after vascular injury or stent implantation, and angioplasty (neointima formation, smooth muscle cell migration and proliferation),

acute kidney failure,

angiogenesis-associated microangiopathies such as, for example, diabetic angiopathies or retinopathy or rheumatoid arthritis,

blood platelet-mediated vascular occlusion, arterial thrombosis,

stroke, reperfusion damage after myocardial infarct or stroke,

cancers, such as, for example, in tumor metastasis or in tumor growth (tumor-induced angiogenesis),

osteoporosis (bone resorption after chemotaxis and adhesion of osteoclasts to bone matrix),

high blood pressure, psoriasis, hyperparathyroidism, Paget's disease, malignant hypercalcemia, metastatic osteolytic lesions, inflammation, wound-healing, cardiac insufficiency, congestive heart failure CHF, and in

antiviral, antimycotic, antiparasitic or antibacterial therapy and prophylaxis (adhesion and internalization).

Advantageously, the compounds of the formula I can be administered in combination with at least one further compound in order to achieve an improved curative action in a number of indications. These further compounds can have the same or a different mechanism of action than the compounds of the formula I.

In addition to the compounds of the formula I and the customary pharmaceutical excipients, the pharmaceutical preparations can therefore contain at least one further compound, depending on the indication, in each case selected from one of the 10 groups below.

Group 1:

inhibitors of blood platelet adhesion, activation or aggregation, such as, for example, acetylsalicylic acid, lysine acetylsalicylate, pilacetyme, dipyridamol, abciximab, thromboxane antagonists, fibrinogen antagonists, such as, for example, tirofiban, or inhibitors of ADP-induced aggregation such as, for example, ticlopidine or clopidogrel, anticoagulants which prevent thrombin activity or formation, such as, for example,

inhibitors of IIa, Xa, XIa, IXa or VIIa,

antagonists of blood platelet-activating compounds and

selectin antagonists

for the treatment of blood platelet-mediated vascular occlusion or thrombosis, or

Group 2:

inhibitors of blood platelet activation or aggregation, such as, for example, GPIIb/IIIa antagonists, thrombin or factor Xa inhibitors or ADP receptor antagonists,

serine protease inhibitors,

fibrinogen-lowering compounds,

selectin antagonists,

antagonists of ICAM-1 or VCAM-1

inhibitors of leukocyte adhesion

inhibitors of vessel wall transmigration,

fibrinolysis-modulating compounds, such as, for example, streptokinase, tPA, plasminogen activation stimulants, TAFI inhibitors, XIa inhibitors or PAI-1 antagonists, inhibitors of complement factors,

endothelin receptor antagonists,

tyrosine kinase inhibitors,

antioxidants and

interleukin 8 antagonists

for the treatment of myocardial infarct or stroke, or

Group 3:

endothelin antagonists,

ACE inhibitors,

angiotensin receptor antagonisten,

endopeptidase inhibitors,

beta-blockers,

calcium channel antagonists,

phosphodiesterase inhibitors and

caspase inhibitors

for the the treatment of congestive heart failure, or

Group 4:

thrombin inhibitors,

inhibitors of factor Xa,

inhibitors of the coagulation pathway which leads to thrombin formation, such as, for example, heparin or low molecular weight heparins,

inhibitors of blood platelet adhesion, activation or aggregation, such as, for example,

GPIIb-IIIa antagonists or antagonists of the blood platelet adhesion and activation mediated by vWF or GPIb,

endothelin receptor antagonists,

nitrogen oxide synthase inhibitors,

CD44 antagonists,

selectin antagonists,

MCP-1 antagonists,

inhibitors of signal transduction in proliferating cells,

antagonists of the cell response mediated by EGF, PDGF, VEGF or bFGF and antioxidants

for the treatment of restenosis after vascular injury or stent implantation, or

Group 5:

antagonists of the cell response mediated by EGF, PDGF, VEGF or bFGF, heparin or low molecular weight heparins or further GAGs,

inhibitors of MMPs,

selectin antagonists,

endothelin antagonists,

ACE inhibitors,

angiotensin receptor antagonists and

glycosylation inhibitors or AGE formation inhibitors or AGE breakers and antagonists of their receptors, such as, for example, RAGE,

for the treatment of diabetic angiopathies or

Group 6:

lipid-lowering compounds,

selectin antagonists,

antagonists of ICAM-1 or VCAM-1

heparin or low molecular weight heparins or further GAGs,

inhibitors of MMPs,

endothelin antagonists,

apolipoprotein A1 antagonists,

cholesterol antagonists,

HMG CoA reductase inhibitors,

ACAT inhibitors,

ACE inhibitors,

angiotensin receptor antagonists,

tyrosine kinase inhibitors,

protein kinase C inhibitors,

calcium channel antagonists,

LDL receptor function stimulants,

antioxidants

LCAT mimetics and

free radical scavengers

for the treatment of atherosclerosis or

Group 7:

cytostatic or antineoplastic compounds,

compounds which inhibit proliferation, such as, for example, kinase inhibitors and heparin or low molecular weight heparins or further GAGs

for the treatment of cancer, preferably for the inhibition of tumor growth or metastasis, or

Group 8:

compounds for anti-resorptive therapy,

compounds for hormone replacement therapy, such as, for example, estrogen or progesterone antagonists,

recombinant human growth hormone,

bisphosphonates, such as, for example, alendronates

compounds for calcitonin therapy,

calcitonin stimulants,

calcium channel antagonists,

bone formation stimulants, such as, for example, growth factor agonists,

interleukin-6 antagonists and

Src tyrosine kinase inhibitors

for the treatment of osteoporosis or

Group 9:

TNF antagonists,

antagonists of VLA-4 or VCAM-1,

antagonists of LFA-1, Mac-1 or ICAMs,

complement inhibitors,

immunosuppressants,

interleukin-1, -5 or -8 antagonists and

dihydrofolate reductase inhibitors

for the treatment of rheumatoid arthritis or

Group 10:

collagenase,

PDGF antagonists and

MMPs

for improved wound-healing.

A pharmaceutical preparation comprising at least one compound of the formula I, if appropriate pharmaceutical excipients and at least one further compound, depending on the indication, in each case selected from one of the above groups, is understood as meaning a combined administration of at least one of the compounds of the formula I with at least one further compound, in each case selected from one of the groups described above and, if appropriate, pharmaceutical excipients.

Combined administration can be carried out by means of a substance mixture comprising at least one compound of the formula I, if appropriate pharmaceutical excipients and at least one further compound, depending on the indication, in each case selected from one of the above groups, but also spatially and temporally separate.

In the case of spatially and/or temporally separate administration, the administration of the components of the pharmaceutical preparation, the compounds of the formula I and the compounds selected from one of the abovementioned groups, takes place spatially and/or temporally separately.

For the treatment of restenosis after vascular injury or stenting, the administrations of the compounds of the formula I can take place alone or in combination with at least one compound selected from group 4 locally to the affected sites. It may also be advantageous to coat the stents with these compounds.

For the treatment of osteoporosis, it may be advantageous to carry out the administration of the compounds of the formula I in combination with an antiresorptive or hormone replacement therapy.

The invention accordingly relates to the use of the abovementioned pharmaceutical preparations for the production of medicaments for the treatment of diseases.

In a preferred embodiment, the invention relates to the use of the above-mentioned combined pharmaceutical preparations for the production of medicaments for the treatment of

blood platelet-mediated vascular occlusion or thrombosis

when using compounds of group 1,

myocardial infarct or stroke

when using compounds of group 2,

congestive heart failure

when using compounds of group 3,

restenosis after vascular injury or stent implantation

when using compounds of group 4,

diabetic angiopathies

when using compounds of group 5,

atherosclerosis

when using compounds of group 6,

cancer

when using compounds of group 7,

osteoporosis

when using compounds of group 8,

rheumatoid arthritis

when using compounds of group 9,

wound-healing

when using compounds of group 10.

The invention is illustrated with the aid of examples below.

### I. SYNTHESIS EXAMPLES

I.1. Precursors

Methyl (2S)-3-[(tert-butoxycarbonyl)amino]-2-{[(dimethylamino)sulfonyl]amino}-propionate (1)

10 g of H-DAP(Boc)-OMe×HCl (39.29 mmol)—Bachem—in 150 ml of CH₂Cl₂ were treated with 15 ml of triethylamine and 15 g of dimethylaminosulfamoyl chloride—dissolved in 50 ml CH₂Cl₂— were added dropwise at 5° C. over the course of 20 min and the mixture was stirred at RT overnight. On the next day, 2 g of dimethylaminosulfamoyl chloride were again added, and the mixture was stirred for 3 h at RT, diluted with CH₂Cl₂ and washed successively with 5% citric acid and saturated NaCl solution. Drying over Na₂SO₄, filtering and concentration afforded 12.7 g of a slightly yellow oil.

ESI-MS: 270.05 [M+H⁺-tBu], 348.1 [M+Na⁺].

¹H-NMR (400 MHz, DMSO) δ ppm: 7.65 (d, 1H), 6.95 (t, 1H), 3.80 (m, 1H), 3.20 (m, 2H), 2.70 (s, 6H), 1.30 (s, 9H).

Methyl (2S)-2-{[(dimethylamino)sulfonyl]amino}-3-(5-methyl-2-oxo-4-sulfanyl-1(2H)-pyrimidinyl)propionate (2)

a.) 11.1 g of methyl (2S)-3-[(tert-butoxycarbonyl)amino]-2-{[(dimethylamino)sulfonyl]-amino}propionate (1) in 100 ml of CH₂Cl₂ were treated with 25 ml of 4N HCl in dioxane and the mixture was stirred for 3 h at RT. Concentration afforded 8.6 g of a clear oil, which was reacted further without further purification.

b.) 3.8 g of 2-(N-carbethoxythiocarbamoyl)-1-(N-piperidino)-1-propene (14.8 mmol; preparation according to WO 00/61551 or J. Heterocycl. Chem. 1968, 5, 837-844) and 3.4 g of methyl (2S)-3-amino-2-{[(dimethylamino)sulfonyl]amino}propionate (hydrochloride) in 150 ml of CH₃OH were treated with 5.1 ml of N-methylmorpholine and the mixture was stirred at RT overnight. For work-up, the mixture was concentrated, taken up in CH₂Cl₂, washed with 1 N HCl and saturated NaCl solution, dried and concentrated. The crude product thus obtained (6.4 g of red oil) was purified by chromatography on silica gel (CH₂Cl₂/CH₃OH 1-5%).

2.2 g of yellow foam; ESI-MS [M+H⁺]: 351.05.

¹H-NMR (360 MHz, DMSO) δ ppm: 9.80 (s, 1H), 7.20 (s, 1H), 5.55 (d, 1H), 4.40 (m, 1H), 4.20 and 4.05 (each dd, 1H), 3.85 (s, 3H), 2.80 (s, 6H), 2.15 (s, 3H).

Methyl (2S)-3-(4-(cyanosulfanyl)-5-methyl-2-oxo-1(2H)-pyrimidinyl)-2-{[(dimethylamino)sulfonyl]amino}propionate (3)

1.5 g of methyl (2S)-2{[(dimethylamino)sulfonyl]amino}-3-(5-methyl-2-oxo-4-sulfanyl-1 (2H)-pyrimidinyl)propionate (component 2b; 4.28 mmol) in 90 ml of CH₂Cl₂ were treated at RT with 1.4 g of KCN— dissolved in 30 ml of aqueous 5% NaHCO₃—and 0.01 g of 18-crown-6, and then at 0° C. 0.45 g of BrCN—dissolved in 10 ml of CH₂Cl₂—was added dropwise. The reaction was complete after about 10 minutes (TLC CH₂Cl₂/acetone 6:1). For work-up, the phases were separated, and the org. phase was washed with H₂O, dried and concentrated. 1.44 g of a brown-yellow foam were isolated, which was employed further without further purification.

Methyl (2S)-2-{[(dimethylamino)sulfonyl]amino}-3-(2-oxo-5-(2-phenylethyl)-4-sulfanyl-1(2H)-pyrimidinyl)propionate (4)

Analogously to component 2b, starting from 10 g of 2-(N-carbethoxythiocarbamoyl)-1-(N-morpholin-4-yl)-4-phenyl-but-1-ene (preparation as in WO 00/61551 starting from phenylbutyraldehyde). After stirring the oily crude product with methyl tert-butyl ether,

9.4 g of a yellow solid were obtained; ESI-MS [M+H⁺]: 441.15.

¹H-NMR (400 MHz, DMSO) δ ppm: 7.99 (d, 1H), 7.55 (s, 1H), 7.35-7.15 (, 5H), 4.25-4.15 (m, 2H), 3.75 (s, 3H), 3.65 (m, 1H), 2.80-2.65 (m, 4H), 2.60 (s, 6H).

Methyl (2S)-3-(4-(cyanosulfanyl)-2-oxo-6-(2-phenylethyl)-1(2H)-pyrimidinyl)-2-{[(dimethylamino)sulfonyl]amino}propionate (5)

Preparation analogously to component 3; 2.6 g of brown-yellow foam, which was reacted directly without further purification.

Ethyl (2S)-3-[(tert-butoxycarbonyl)amino]-2-{[(dimethylamino)sulfonyl]amino}-propionate (6)

A solution of 6.7 g of H-DAP(Boc)-OEt×HCl (J. Labelled Radiopharm. 1999, 42, 605-609; 25 mmol), 3.95 g (27.5 mmol) of dimethylsulfamoyl chloride, 5.8 g of triethylamine and 180 mg of 4-dimethylaminopyridine in 150 ml of CH₂Cl₂ was refluxed for 25 h (TLC: CH₂Cl₂/acetone/CH₃OH 45/5/0.5). The reaction solution was then washed with H₂O, 5% citric acid solution, H₂O and 5% NaHCO₃ solution, dried using Na₂SO₄ and evaporated. The oily residue was dissolved in 10 ml of diethyl ether, mixed with 3 ml of water-saturated ether, cooled to 0° C., where crystallization commenced, which was then completed by addition of n-hexane. After filtering off with suction and washing with diethyl ether/n-hexane (1/4), 6.2 g of white crystals were isolated.

M.p.: 65-67° C.; ESI-MS [M+H⁺]: 340.

Ethyl (2S)-3-(4-(cyanosulfanyl)-5-methyl-2-oxo-1(2H)-pyrimidinyl)-2-{[(dimethyl-amino)sulfonyl]amino}propionate (7)

a.) 6.0 g of the above compound were dissolved in 30 ml of CH₂Cl₂ and, after addition of 20 ml of TFA, stirred overnight at RT. After stripping off the solvent, the residue was taken up several times in CH₂Cl₂, treated with n-hexane until the occurence of turbidity and the solvent was removed by distillation again. The residue thus obtained was employed in the subsequent reactions without further purification.

b.) 4.15 g (16 mmol) of 2-(N-carbethoxythiocarbamoyl)-1-(N-piperidino)-1-propene were added at 10° C. to a solution of 6.2 g (17.7 mmol) of the trifluoroacetate and 3.0 g of diisopropylethylamine in 40 ml of ethanol and the mixture was stirred for 2 h at RT. For work-up, it was adjusted to pH 3 using 4N HCl in dioxane, the ethanol was removed by distillation, the residue was taken up in a mixture of 150 ml of ethyl acetate and 50 ml of diethyl ether, and the org. phase was washed with H₂O, 5% citric acid solution, 5% NaHCO₃ solution and saturated NaCl solution, dried and concentrated. The viscous orange-red residue was dissolved in 40 ml of toluene with addition of 15 ml of ethyl acetate in the presence of heat, and the voluminous precipitate depositing on cooling was converted into a form which could be filtered off with suction by fresh addition of 30 ml of toluene and 5 ml of ethyl acetate. 5.2 g of orange-coloured crystals were isolated.

M.p.: 158° C.; ESI-MS [M+H⁺]: 365.

Ethyl (2S)-3-(4-(cyanosulfanyl)-5-methyl-2-oxo-1(2H)-pyrimidinyl)-2-{[(dimethylamino)sulfonyl]amino}propionate (8)

Procedure analogous to the preparation of component 3; 5.3 g of yellow amorphous residue; ESI-MS [M−H⁺]: 390.

7-(4-Piperidinyl)-1,2,3,4-tetrahydro[1,8]naphthyridine (9)

a.) A solution of 1-(tert-butoxycarbonyl)-4-piperidinecarboxylic acid (110.0 mmol, 25.0 g), O,N-dimethylhydroxylamine hydrochloride (110.0 mmol, 10.63 g), N-methylmorpholine (0.75 mol, 75.85 g), HOBT (140.0 mmol, 21.05 g) and EDC×HCl (140.0 mmol, 26.35 g) in CH₃CN (500 ml) was stirred overnight at RT. After evaporation, the residue was taken up in ethyl acetate, washed successively with H₂O, a 10% KHSO₄ soln, sat. aq. NaHCO₃ solution and sat. aq. NaCO₃ solution. Drying and evaporation of the org. phase afforded 23.60 g of yellowish oil; ESI-MS: [M+Na⁺]=295, [M-tBu+H⁺]=217.05.

b.) Methylmagnesium bromide (190.0 mmol, 53.0 ml of a 3M solution in Et₂O) was added dropwise at 0° C. to a solution of tert-butyl 4-{[methoxy(methyl)amino]carbonyl}-1-piperidinecarboxylate (9a, 80.0 mmol, 23.1 g) in THF (300 ml) and the mixture was stirred for 2 h at 0° C. The reaction mixture was then cautiously acidified with a 10% KHSO₄ soln (50 ml), extracted with ethyl acetate and the org. phase was then washed with sat. aq. NaHCO₃ and sat. aq. NaCl solution, dried and evaporated: 19.1 g of yellowish oil; ESI-MS: [M-tBu+H⁺]=172.1.

c.) A mixture of tert-butyl 4-acetyl-1-piperidinecarboxylate (9b, 81.39 mmol, 18.5 g), 2-aminonicotinaldehyde (Heterocycl. 1993, 36, 2518; 92.78 mmol, 11.33 g) and KOH (3.50 ml of a 20% aq. soln) in ethanol (240.0 ml) was heated to reflux for 8 h. After evaporation, the residue was taken up in methylene chloride and washed with water; evaporation of the org. phase afforded 25.10 g of the target product; ESI-MS: [M+H⁺]=314.1, [M-Boc+H⁺]=214.1.

d.) A suspension of tert-butyl 4-[1,8]naphthyridin-2-yl-1-piperidinecarboxylate (79.13 mmol, 24.80 g) and Pd/C (10%, 4.0 g) in ethanol (200 ml) was stirred at RT overnight under an H₂ atmosphere, then filtered through Celite and washed with ethanol. Evaporation afforded 18.6 g; ESI-MS: [M+H⁺]=318.25.

¹H-NMR (360 MHz, CDCl₃) δ (ppm): 7.07 (1H, d), 6.31 (1H, d), 4.87 (1H, s br.), 4.19 (2H, s br.), 3.38 (2H, m sym.), 2.77 (2H, t br.), 2.68 (2H, t), 2.57 (1H, tt), 1.93-1.80 (4H, m), 1.61 (2H, qd), 1.45 (9H, s).

e.) TFA (560.0 mmol, 64.19 g) was added to a solution of tert-butyl 4-(5,6,7,8-tetra-hydro[1,8]naphthyridin-2-yl)-1-piperidinecarboxylate (9d, 60.0 mmol, 18.0 g) in CH₂Cl₂ (400 ml), and the solution was stirred for 20 h and evaporated; the residue was dissolved in in NH₄OH (75 ml) and extracted exhaustively with ethyl acetate (3×250 ml); evaporation of the org. phase afforded 10.20 g; ESI-MS: [M+H⁺]=218.5, 109.7.

¹H-NMR (400 MHz, CDCl₃) δ (ppm): 7.09 (1H, d), 6.35 (1H, d), 4.84 (1H, s br.), 3.40 (2H, t), 3.19 (2H, d), 2.95 (2H, s), 2.81-2.65 (4H, m), 2.55 (1H, m sym.), 1.97-1.82 (4H, m), 1.72-1.56 (2H, m).

I.2 Compounds of the Formula (I)

### Example 1

Ethyl (2S)-3-(4-{4-[(1H-benzimidazol-2-ylamino)methyl]-1-piperidinyl}-5-methyl-2-oxo-1(2H)-pyrimidinyl)-2-{[(dimethylamino)sulfonyl]amino}propionate

A suspension of 6.35 g (13.9 mmol) of N-(1H-benzimidazol-2-yl)-N-(4-piperynylmethyl)-amine bistrifluoroacetate in 50 ml of CH₂Cl₂ and 10 ml of acetonitrile was dissolved by addition of 8.0 g of diisopropylethylamine, then a solution of 5.3 g (13.6 mmol) of ethyl (2S)-3-(4-(cyanosulfanyl)-5-methyl-2-oxo-1 (2H)-pyrimidinyl)-2{[(dimethylamino)-sulfonyl]amino}propionate (8) in 20 ml of CH₂Cl₂ was added dropwise and the mixture was stirred for 2 h at RT. For work-up, the reaction solution was washed with H₂O, 5% NaHCO₃ and saturated NaCl solution, dried and the solvent was removed by distillation. By digestion with ethyl acetate with addition of a little acetone, the residue was converted into a crystalline state. Chromatography twice on silica gel (eluent: CH₂Cl₂/ethanol/acetic acid 5/4/1), removal of the eluent by distillation, dissolution in CH₂Cl₂/CH₃OH (95/5), extraction by shaking with 5% NaHCO₃ solution, stripping off the solvent and digesting the residue with isopropanol afforded 4.3 g as a slightly yellowish powder.

M.p.: 200-202° C. (dec.); ESI-MS [M−H⁺]: 561.

¹H-NMR (360 MHz, DMSO) δ ppm: 10.6 (s, 1H), 7.45 (s, 1H), 7.15 (m, 1H), 6.90, 6.80 and 6.70 (in each case m, 1H), 4.25-4.10 (m, 6H), 3.60 (dd, 1H), 3.20 and 2.85 (each m, 2H), 2.55 (s, 6H), 2.05 (s, 3H), 1.90 (m, 1H), 1.80 (m, 2H), 1.20 (m, 5H).

### Example 2

(2S)-3-(4-{4-[(1H-Benzimidazol-2-ylamino)methyl]-1-piperidinyl}-5-methyl-2-oxo-1 (2H)-pyrimidinyl)-2-{[(dimethylamino)sulfonyl]amino}propionic acid (dihydrochloride)

a.) 22.1 g (35 mmol) of 2-[({1-[1-((2S)-2-{[(benzyloxy)carbonyl]amino}-2-carboxyethyl)-5-methyl-2-oxo-1,2-dihydropyrimidin-4-yl]piperidin-4-yl}methyl)amino]-1H-benzimidazol-1-ium dihydrochloride (WO 00/61551; preparation analogously to the corresponding acetate Example 1-72) were suspended in 200 ml of a 30% HBr solution in acetic acid and stirred under nitrogen for 3 h at RT. According to TLC, starting material was no longer detectable in the dark-yellow reaction solution (TLC CH₂Cl₂/CH₃OH/50% acetic acid 7/3/1). For work-up, the mixture was poured into 2.5 l of diethyl ether, the precipitate depositing was decanted off from the supernatant ether, the residue was suspended again and the decantation process was repeated several times. The fine precipitate was filtered off with suction, washed well with diethyl ether and finally n-hexane, dissolved in still-moist form in 500 ml of ethanol and adjusted to a pH of 5-6 using diisopropylethyl-amine, a voluminous thick precipitate depositing, which was filtered off with suction and washed with ethanol. This residue was again suspended in 500 ml of ethanol, treated with diisopropylethylamine (pH about 9) and allowed to stand at RT for about 50 h, a form which could readily be filtered off with suction being formed, which, after filtering off with suction and washing with ethanol and diethyl ether, was dried in a vacuum drying oven at 50° C. 14.5 g of (2S)-2-amino-3-(4-{4-[(1H-benzimidazol-2-ylamino)methyl]-1-piperidinyl}-5-methyl-2-oxo-1 (2H)-pyrimidinyl)propionic acid were isolated as a fine powder; ESI-MS [M−H⁺]: 426.

b.) 12 ml of 1 N NaOH and a solution of 1.73 g (12 mmol) of dimethylaminosulfamoyl chloride in 5 ml of dioxane were simultaneously added dropwise to a solution of 1.7 g of the above compound in a mixture of 40 ml of dioxane, 4 ml of H₂O and 6 ml of 1 N NaOH with stirring over the course of 7 h and the mixture was then stirred overnight at RT. The reaction solution was then adjusted to pH 2.5 using 1 N HCl, the solvent was largely stripped off, the residue was digested several times with ethanol, NaCl meanwhile deposited was filtered off, the combined ethanol extracts were evaporated and the residue was purified by chromatography (eluent: CH₂Cl₂/CH₃OH/NH₃ 35/15/4). After stripping off the eluent, the residue was taken up in 100 ml of H₂O and freeze dried after addition of 2 equivalents of 1 N HCl. 1.9 g of white amorphous solid; ESI-MS [M−H⁺]: 533.

¹H-NMR (360 MHz, DMSO) δ ppm: 9.3 (broad s, 1H), 8.85-8.70 (m, 2H), 7.30 and 7.20 (in each case m, 2H), 4.30-4.15 (m, 4H), 3.70 (m, 2H, superimposed by H₂O), 3.20 (m, 2H), 2.60 (s, 6H), 2.15 (s, 3H), 2.05 (m, 1H), 1.90 (m, 2H), 1.35 (m, 2H).

### Example 3

(2S)-3-(4-[4-(1H-Benzimidazol-2-ylmethyl)-1-piperidinyl]-2-oxo-5-(2-phenylethyl)-1 (2H)-pyrimidinyl)-2-{[(dimethylamino)sulfonyl]amino}propionic acid

0.6 g (1.29 mmol) of methyl-(2S)-3-(4-(cyanosulfanyl)-2-oxo-5-(2-phenylethyl)-1 (2H)-pyrimidinyl)-2-{[(dimethylamino)sulfonyl]amino}propionate (5), 0.58 g of 2-(4-piperidinyl-methyl)-1H-benzimidazole (bistrifluoroacetate), 0.5 g of molecular sieve 3 A and 0.72 g of K₂CO₃ were stirred overnight at RT in 50 ml of dry acetonitrile. For work-up, the mixture was filtered, and the filtrate was diluted with CH₂Cl₂, washed with saturated NaCl solution, dried and concentrated: 0.77 g of yellow foam, ESI-MS [M+H⁺]: 622.15. The isolated crude product was dissolved in a mixture of 20 ml of dioxane and 10 ml of H₂O and, after addition of 100 mg of KOH, stirred for 3 h at RT (TLC CH₂Cl₂1CH₃OH/acetic acid 9/1/0.1). The reaction mixture was concentrated, stirred with 20 ml of 50% acetic acid, and the resulting solid was filtered off with suction, washed thoroughly with H₂O and then dried in a vacuum drying oven. 0.53 g of yellowish amorphous solid; ESI-MS [M+H⁺]: 608.3.

¹H-NMR (360 MHz, DMSO) δ ppm: 7.65 (d, 1H), 7.60 (s, 1H), 7.50 (m, 2H), 7.35-7.05 (m, 7H), 4.30-4.15 (m, 2H), 4.05 (m, 2H), 3.50 (m, 1H; superimposed with H₂O), 2.95-2.65 (m, 8H), 2.60 (s, 6H), 2.15 (m, 1H), 1.75 and 1.25 (each m, 2H).

The following were prepared analogously to example 3:

### Example 4

(2S)-3-(4-{4-[(1H-Benzimidazol-2-ylamino)methyl]-1-piperidinyl}-2-oxo-5-(2-phenylethyl)-1 (2H)-pyrimidinyl)-2-{[(dimethylamino)sulfonyl]amino}propionic acid Reaction of 5 with N-(1H-benzimidazol-2-yl)-N-(4-piperidinylmethyl)amine (WO 00/61551). 650 mg; ESI-MS [M+H⁺]: 637.3. Cleavage of the methyl ester afforded 270 mg of ocher-coloured amorphous solid; ESI-MS [M+H⁺]: 623.35.

¹H-NMR (360 MHz, DMSO) δ ppm: 7.85 (m, 1H), 7.65 (s, 1H), 7.45 (m, 1H), 7.35-7.20 (m, 8H), 7.0 (m, 2H), 4.25 (dd, 1H), 4.15 (m, 2H), 3.45 (dd, 1H), 2.85-2.50 (m, 8H), superimposed with 2.60 (s, 6H), 1.85 (m, 1H), 1.80 (m, 2H), 1.20 (m, 2H).

### Example 5

(2S)-2-{[(Dimethylamino)sulfonyl]amino}-3-(2-oxo-5-(2-phenylethyl)₄-{4-[(2-pyridinylamino)methyl]-1-piperidinyl}-1 (2H)-pyrimidinyl)propionic acid

Reaction of 5 with N-(piperidin-4-ylmethyl)pyridin-2-amine (WO 00/61551). 780 mg; ESI-MS [M+H⁺]: 598.35. Cleavage of the methyl ester, purification of the crude product by means of MPLC (silica gel: Bischoff Prontoprep 60-2540-Cl 8E, 32 μm; mobile phase: CH₃CN/H₂O+0.1% acetic acid) and subsequent freeze drying afforded 175 mg of white amorphous solid; ESI-MS [M+H⁺]: 623.35.

¹H-NMR (360 MHz, DMSO) δ ppm: 8.05 (d, 1H), 7.95 (d, 1H) superimposed with 7.85 (s, 1H), 7.45-7.25 (m, 6H), 6.70 (m, 1H), 6.60 (m, 2H), 4.35 (dd, 1H), 4.30 (m, 1H), 4.20 (m, 2H), 3.65 (dd, 1H, superimposed by H₂O), 3.25 (m, 2H), 3.0-2.70 (m, 6H), 2.60 (s, 6H), 1.80 (m, 3H), 1.25 (m 2H).

### Example 6

(2S)-2-{[(Dimethylamino)sulfonyl]amino}-3-(2-oxo-5-(2-phenylethyl)-4-[4-(5,6,7,8-tetrahydro[1,8]naphthyridin-2-yl)-1-piperidinyl]-1 (2H)-pyrimidinyl)propionic acid

Reaction of 5 with 6-(4-piperidinyl)-1,2,3,4-tetrahydro[1,8]naphthyridine (9). 720 mg; ESI-MS [M+H⁺]: 624.35. Cleavage of the methyl ester, purification of the crude product by chromatography on silica gel (CH₂Cl₂/CH₃OH 5%+1% acetic acid) and subsequent freeze drying afforded 270 mg of white amorphous solid; ESI-MS [M+H⁺]: 610.35.

¹H-NMR (360 MHz, DMSO) δ ppm: 7.65 (s, 1H), 7.35-7.20 (m, 5H), 7.10 (s, 1H), 6.30 (m, 2H), 4.30 (dd, 1H), 4.15 (m, 2H), 3.95 (dd, 1H), 3.60-3.20 (m, superimposed with H₂O), 2.95 (m, 2H), 2.85 (m, 2H), 2.65 (m, 4H), 2.60 (s, 6H), 1.80 (m, 5H).

### Example 7

(2S)-3-(4-[4-(1H-Benzimidazol-2-ylmethyl)-1-piperidinyl]-5-methyl-2-oxo-1 (2H)-pyrimidinyl)-2-{[(dimethylamino)sulfonyl]amino}propionic acid (acetate)

Reaction of 3 with 2-(4-piperidinylmethyl)-1H-benzimidazole (bistrifluoroacetate) afforded 640 mg; ESI-MS [M+H⁺]: 532.3. Cleavage of the methyl ester, purification of the crude product by chromatography on silica gel (CH₂Cl₂/CH₃OH 5%+1% acetic acid) and subsequent freeze drying afforded 350 mg of amorphous yellow solid; ESI-MS [M+H⁺]: 518.15.

¹H-NMR (360 MHz, DMSO) δ ppm: 7.45 and 7.10 (each m, 2H), 4.25 (dd, 1H), 4.15 (m, 2H), 3.80 (dd, 1H), 3.35 (dd, 1H), 2.85-2.75 (m, 4H), 2.60-2.50 (m superimposed, 2H), 2.60 (s, 6H), 2.20 (m, 1H), 2.10 (s, 3H), 1.95 (s, 3H), 1.25 (m, 2H).

### Example 8

(2S)-2{[(Dimethylamino)sulfonyl]amino}-3-(5-methyl-2-oxo-4-[4-(5,6,7,8-tetrahydro[1,8]naphthyridin-2-yl)-1-piperidinyl]-1 (2H)-pyrimidinyl)propionic acid

Reaction of 3 with 6-(4-piperidinyl)-1,2,3,4-tetrahydro[1,8]naphthyridine (9) afforded 730 mg; ESI-MS [M+H⁺]: 534.25. Cleavage of the methyl ester, purification of the crude product by chromatography by means of MPLC (silica gel: Bischoff Prontoprep 60-2540-C18E, 32 μm; mobile phase: CH₃CN/H₂O+0.1% acetic acid) and subsequent freeze drying afforded 420 mg of white amorphous solid; ESI-MS [M+H⁺]: 520.25.

¹H-NMR (360 MHz, DMSO) δ ppm: 7.60 (d, 1H), 7.45 (s, 1H), 7.15 (d, 1H), 6.65 (m, 1H), 6.30 (d, 1H), 4.20 (m, 3H), 3.75-3.40 (m superimposed with H₂O), 3.25 (m, 2H), 2.95 (m, 2H), 2.70 (m, 1H), 2.65 (m, 2H), 2.45 (s, 6H), 2.05 (s, 3H), 1.80-1.60 (m, 4H).

### Example 9

(2S)-2-{[(Dimethylamino)sulfonyl]amino}-3-(5-methyl-2-oxo-4-{4-[(2-pyridinylamino)-methyl]-1-piperidinyl}-1 (2H)-pyrimidinyl)propionic acid

Reaction of 3 with N-(piperidin-4-ylmethyl)pyridin-2-amine (WO 00/61551) afforded 570 mg of yellow solid; ESI-MS [M+H⁺]: 508.25. Cleavage of the methyl ester, purification of the crude product by means of MPLC (silica gel: Bischoff Prontoprep 60-2540-C₁₈E, 32 μm; mobile phase: CH₃CN/H₂O+0.1% acetic acid) and subsequent freeze drying afforded 340 mg of amorphous white solid; ESI-MS [M+H⁺]: 494.25.

¹H-NMR (360 MHz, DMSO) δ ppm: 7.95 (d, 1H), 7.65 (d, 1H), 7.40 (s, 1H), 7.35 (m, 1H), 6.65 (m, 1H), 6.25-6.20 (m, 2H), 4.25 (m, 1H), 4.20 (m, 2H), 3.33 and 3.45 (each dd, 1H), 3.20 (m, 2H), 2.85 (m, 2H), 2.55 (s, 6H), 2.10 (s, 3H), 1.80 (m, 3H), 1.20 (m, 2H).

### Example 10

Ethyl (2S)-3-(4-[4-(1H-benzimidazol-2-ylmethyl)-1-piperidinyl]-5-methyl-2-oxo-1 (2H)-pyrimidinyl)-2-{[(dimethylamino)sulfonyl]amino}propionate

130 mg (0.23 mmol) of (2S)-3-(4-[4-(1H-benzimidazol-2-ylmethyl)-1-piperidinyl]-5-methyl-2-oxo-1 (2H)-pyrimidinyl)-2-{[(dimethylamino)sulfonyl]amino}propionic acid (acetate), Example 7, were dissolved in 20 ml of ethanol, 1 ml of ethereal HCl (saturated at 0° C.) was added, and the mixture was stirred overnight at RT and then for 10 h at 50° C. (TLC CH₂Cl₂/CH₃OH/NH₄OH 2015/0.5). The reaction mixture was evaporated and the oil obtained was freeze dried. 97 mg of yellowish solid.

¹H-NMR (360 MHz, DMSO) δ ppm: 7.95 (d, 1H), 7.80 (m, 2H), 7.70 (s, 1H), 7.50 (d, 2H), 4.30-4.10 (m, 6H), 3.70 (m, 1H), 3.20 (m, 2H), 3.05 (m, 2H), 2.60 (s, 6H), 2.35 (m, 1H), 2.05 (s, 3H), 1.80 (m, 2H), 1.40 (m, 2H), 1.25 (t, 3H).

### Example 11

Ethyl (2S)-2-{[(dimethylamino)sulfonyl]amino}-3-(5-methyl-2-oxo-4-[4-(5,6,7,8-tetrahydro[1,8]naphthyridin-2-yl)-1-piperidinyl]-1 (2H)-pyrimidinyl)propionate

180 mg (0.35 mmol) of (2S)-2-{[(dimethylamino)sulfonyl]amino}-3-(5-methyl-2-oxo-4-[4-(5,6,7,8-tetrahydro[1,8]naphthyridin-2-yl)-1-piperidinyl]-1 (2H)-pyrimidinyl)propionic acid, Example 8, were converted into the ethyl ester analogously to Example 10. 90 mg; ESI-MS [M+H⁺]: 548.35.

¹H-NMR (360 MHz, DMSO) δ ppm: 7.85 (d, 1H), 7.45 (s, 1H), 7.20 (m, 1H), 6.30 (d, 1H), 4.25 (m, 2H), 4.15 (m, 2H), 3.65 (dd, 1H), 3.25 (m, 1H), 2.95 (m, 2H), 2.75 (m, 1H), 2.65 (m, 2H), 2.5 (s, 6H), 2.05 (s, 3H), 1.80-1.60 (m, 8H), 1.20 (t, 3H).

### II. BIOLOGICAL AND PHARMACOLOGICAL EXAMPLES

### Example 1

Integrin αᵥβ₃ Assay

For the identification and assessment of integrin αᵥβ₃ ligands, a test system was used which is based on competition between the natural integrin αᵥβ₃ ligand vitronectin and the test substance for binding to solid phase-bound integrin αᵥβ₃.

Procedure

coat microtiter plates with 250 ng/ml of integrin αᵥβ₃ in 0.05 M NaHCO₃ pH 9.2; 0.1 ml/well; overnight/4° C.

saturate with 1% milk powder/assay buffer; 0.3 ml/well; 0.5 h/RT

wash 3× with 0.05% Tween 20/assay buffer

test substance in 0.1% milk powder/assay buffer, 50 μl/well +0 μg/ml or 2 μg/ml of human vitronectin (Boehringer Ingelheim T007) in 0.1% milk powder/assay buffer, 50 μl/well; 1 h/RT

wash 3× with 0.05% Tween 20/assay buffer

1 μg/ml of anti-human vitronectin antibody coupled to peroxidase (Kordia SAVN-APHRP) in 0.1% milk powder/assay buffer; 0.1 ml/well; 1 h/RT

wash 3× with 0.05% Tween 20/assay buffer

0.1 ml/well of peroxidase substrate

stop reaction with 0.1 ml/well of 2 M H₂SO₄

measurement of the absorption at 450 nm

Integrin αᵥβ₃: Human placenta is solubilized with Nonidet and integrin αᵥβ₃ is affinity-purified on a GRGDSPK matrix (elution mit EDTA). Impurities due to integrin αᵥβ₃ and human serum albumin, and the detergent and EDTA are removed by anion exchange chromatography.

Assay buffer: 50 mM Tris pH 7.5; 100 mM NaCl; 1 mM CaCl₂; 1 mM MgCl₂; 10 μM MnCl₂

Peroxidase substrate: mix 0.1 ml of TMB solution (42 mM TMB in DMSO) and 10 ml of substrate buffer (0.1 M Na acetate pH 4.9), and then addition of 14.7 μl of 3% H₂O₂.

In the assay, different dilutions of the test substances are employed and the IC₅₀ values are determined (concentration of the ligand at which 50% of the ligand is displaced). The compounds from Examples 2 and 9 showed the best results.

### Example 2

Integrin αᵥβ₃ Assay

The assay is based on competition between the natural integrin α_{IIb}β₃ ligand fibrinogen and the test substance for binding to integrin α_{IIb}β₃.

Procedure

coat microtiter plates with 10 μg/ml of fibrinogen (Calbiochem 341578) in 0.05 M NaHCO₃ pH 9.2; 0.1 ml/well; overnight/4° C.

saturate with 1% BSA/PBS; 0.3 ml/well; 30 min/RT

wash 3× with 0.05% Tween 20/PBS

test substance in 0.1% BSA/PBS; 50 μl/well+200 μg/ml of integrin-α_{IIb}β₃ (Kordia) in 0.1% BSA/PBS; 50 μl/well; 2 to 4 h/RT

wash 3× as above

biotinylated anti integrin α_{IIb}β₃ antibody (Dianova CBL 130 B); 1:1000 in 0.1% BSA/PBS; 0.1 ml/well; 2 to 4 h/RT

wash 3× as above

streptavidin-peroxidase complex (B.M. 1089153) 1:10000 in 0.1% BSA/PBS; 0.1 ml/well; 30 min/RT

wash 3× as above

0.1 ml/well of peroxidase substrate

stop reaction with 0.1 ml/well of 2 M H₂SO₄

measurement of the absorption at 450 nm

Peroxidase substrate: mix 0.1 ml of TMB solution (42 mM TMB in DMSO) and 10 ml of substrate buffer (0.1 M Na acetate pH 4.9), then addition of 14.7 μl of 3% H₂O₂

Different dilutions of the test substances are employed in the assay and the IC₅₀ values are determined (concentration of the antagonist at which 50% of the ligand is displaced). By comparison of the IC₅₀ values in the integrin αᵥβ₃ and integrin αᵥβ₃ assay, the selectivity of the substances can be determined.

### Example 3

CAM Assay

Der CAM (chorioallantoic membrane) assay serves as a generally recognized model for the assessment of the in vivo activity of integrin αᵥβ₃ antagonists. It is based on the inhibition of angiogenesis and neovascularization of tumor tissue (Am. J. Pathol. 1975, 79, 597-618; Cancer Res. 1980, 40, 2300-2309; Nature 1987, 329, 630). The procedure is carried out analogously to the prior art. The growth of the chicken embryo blood vessels and of the transplanted tumor tissue can be readily monitored and assessed.

### Example 4

Rabbit Eye Assay

The inhibition of angiogenesis and neovascularization in the presence of integrin αᵥβ₃-antagonists can be monitored and assessed analogously to Example 3 in this in vivo model. The model is generally recognized and based on the growth of the rabbit blood vessels starting from the edge in the cornea of the eye (Proc. Natl. Acad. Sci. USA. 1994, 91, 40824085; Science 1976, 193, 70-72). The procedure is carried out analogously to the prior art.

### Example 5

Investigation of the Pharmacokinetic Properties in the CACO Model

The experimental procedure is carried out as described by W. Rubas and M. Cromwellin in Advanced Drug Delivery Reviews 23 (1997) 157-162, J. Handler, N. Green and R. Steele in Methods in Enzymology 171 (1989) 736-744 and K. Dharmsathaphom and J. Madara in Methods in Enzymology 192 (1990) 354-370.

The compounds of the general formula I according to the invention are distinguished in comparison to the substances described in WO 00/61551 by more advantageous physicochemical properties, in particular an improved solubility in water. Substances having improved solubility as a rule show markedly increased resorption properties and are thus better orally available. The following table shows a comparison of the solubility of compound Example 2 with structures according to WO 00/61551.

Method:

10 mg of the substance to be investigated were dissolved in 1 ml of DMSO, vigorously shaken for 10 minutes and then centrifuged for 10 minutes at 10000 rpm and diluted with a mixture of 5% CH₃CN/H₂O+0.1% TFA 1:10.

10 mg of substance were mixed with 1 ml of H₂O, vigorously shaken for 10 minutes and centrifuged for 10 minutes at 10000 rpm. The supernatant was removed and likewise diluted. Both solutions were measured by HPLC (column: MACHEREY & NAGEL, Nucleosil C18 PPN, 100×2.1 mm ID, 5μ, temp: 40° C.) and the concentration of the aq. solution was determined.

| | | Solubility | |
|---|---|---|---|
| Substance | (H₂O) | | |
| (2S)-3-(4-{4-[(1H-Benzimidazol-2-ylamino)methyl]-1- | Example 2 | 2.5 | mg/ml |
| piperidinyl}-5-methyl-2-oxo-1(2H)-pyrimidinyl)-2- | | | |
| {[(dimethylamino)sulfonyl]amino}propanoic acid | | | |
| (dihydrochloride) | | | |
| (2S)-3-(4-{4-[(1H-Benzimidazol-2-ylamino)methyl]-1- | WO | <0.05 | mg/ml |
| piperidinyl}-5-methyl-2-oxo-1(2H)-pyrimidinyl)-2- | 00/61551 | | |
| [(phenylsulfonyl)amino]propanoic acid (acetate) | | | |
| (2S)-3-(4-{4-[(1H-Benzimidazol-2-ylamino)methyl]-1- | WO | 0.56 | mg/ml |
| piperidlnyl}-5-methyl-2-oxo-1(2H)-pyrimidinyl)-2- | 00/61651 | | |
| [(ethoxycarbonyl)amino]propanoic acid (hydrochloride) | | | |
| (2S)-3-(4-{4-[(1H-Benzimidazol-2-ylamino)methyl]-1- | WO | 0.86 | mg/ml |
| piperidinyl}-5-methyl-2-oxo-1(2H)-pyrimidinyl)-2- | 00/61551 | | |
| {[(ethylamino)carbonyl]amino}propanoic acid | | | |
| (2S)-3-(4-{4-[(1H-Benzimidazol-2-ylamino)methyl]-1- | WO | 0.81 | mg/ml |
| piperidinyl}-5-methyl-2-oxo-1(2H)-pyrimidinyl)-2- | 00/61551 | | |
| {[(dimethylamino)carbonyl]amino}propanoic acid | | | |
| (hydrochloride) | | | |
| (2S)-3-(4-{4-[(1H-Benzimidazol-2-ylamino)methyl]-1- | WO | <0.05 | mg/ml |
| piperidinyl}-5-methyl-2-oxo-1(2H)-pyrimidinyl)-2- | 00/61551 | | |
| [(methylsulfonyl)amino]propanoic acid (hydrochloride) | | | |
| (2S)-2-(Acetylamino)-3-(4-{4-[(1H-benzimidazol-2- | WO | 1 | mg/ml |
| ylamino)methyl]-1-piperidinyl}-5-methyl-2-oxo-1(2H)- | 00/61551 | | |
| pyrimidinyl)propanoic acid (hydrochloride) | | | |

1. A compound of the general formula (I) 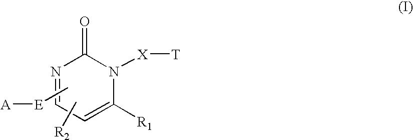
where the radicals have the following meaning:
T=CO₂H, a radical hydrolyzable to CO₂H or a radical isosteric to CO₂H,
X═(CRₓ¹Rₓ²)ₐ-(Gₓ)ₑ-(CRₓ³Rₓ⁴)_{b}-Wₓ—(CRₓ⁵Rₓ⁶)_{c}—(Yₓ)_{f}—(CRₓ⁷Rₓ⁸)_{d}—where
a, b, c, d independently of one another are 0, 1, 2 or 3,
e, f independently of one another are 0 or 1,
the sum of a, b, c, d, e and f is ≦10,
Rₓ¹, Rₓ², Rₓ³, Rₓ⁴, Rₓ⁵, Rₓ⁶, Rₓ⁷, Rₓ⁸ independently of one another are hydrogen, halogen, a hydroxyl group, a branched or unbranched, optionally substituted C₁-C₆-alkyl, C₂-C₆-alkenyl, C₂-C₆-alkynyl or alkylenecycloalkyl radical, a radical —(CH₂)ᵣ—(Yₓ)_{y}—Rₓ⁹, an optionally substituted C₃-C₇-cycloalkyl, aryl, arylalkyl, hetaryl or hetarylalkyl radical, or independently of one another in each case two radicals Rₓ¹ and Rₓ² or Rₓ³ and Rₓ⁴ or Rₓ⁵ and Rₓ⁶ or Rₓ⁷ and Rₓ⁸ together are a 3- to 7-membered, optionally substituted, saturated or unsaturated carbo- or heterocycle, which can contain up to three heteroatoms from the group consisting of O, N and S,
Rₓ⁹ is hydrogen, a hydroxyl group, CN, halogen, a branched or unbranched, optionally substituted C₁-C₆-alkyl, aryl or alkylenearyl radical, a primary or optionally secondary or tertiary substituted amino radical, a C₂-C₆-alkynyl or C₂-C₆-alkenyl radical optionally substituted by C₁-C₄-alkyl or aryl, a C₅-C₁₂-bicycloalkyl or C₈-C₂₀-tricycloalkyl radical, or a 3- to 6-membered, saturated or unsaturated heterocycle substituted by up to three identical or different radicals, which can contain up to three different or identical heteroatoms O, N, S, or a C₃-C₇-cycloalkyl, aryl or heteroaryl radical, where two radicals together can be a fused, saturated, unsaturated or aromatic carbocycle or heterocycle which can contain up to three different or identical heteroatoms O, N, S, and the cycle can be optionally substituted or a further, optionally substituted, saturated, unsaturated or aromatic cycle can be fused to this cycle,
r=0-4
Gₓ and Yₓ independently of one another are CO, CO—NRₓ¹⁰, NRₓ¹⁰CO, S, SO, SO₂, SO₂NRₓ¹⁰, NRₓ¹⁰SO₂, CS, CS—NRₓ¹⁰, NRₓ¹⁰—CS, CS—O, O—CS, CO—O, O—CO, O, ethynyl, CRₓ¹¹—O—CRₓ¹², C(═CRₓ¹¹Rₓ¹²) CRₓ¹¹═CRₓ¹², CRₓ¹¹ (ORₓ¹³)—CRₓ¹², CRₓ¹¹—CRₓ¹²(ORₓ¹³)—,
Rₓ¹⁰ is hydrogen, a branched or unbranched, optionally substituted C₁-C₆-alkyl, alkylenearyl, alkylenealkynyl, hetaryl or alkylenehetaryl radical, an optionally substituted C₃-C₇-cycloalkyl radical, CO—Rₓ¹⁴, COORₓ¹⁴, SO₂—Rₓ¹⁴,
Rₓ¹¹, Rₓ¹² independently of one another are hydrogen, a hydroxyl group, a branched or unbranched, optionally substituted C₁-C₆-alkyl, C₁-C₄-alkoxy, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,
R¹³ is hydrogen, a branched or unbranched, optionally substituted C₁-C₆-alkyl, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,
Rₓ¹⁴ is hydrogen, a hydroxyl group, a branched or unbranched, optionally substituted C₁-C₆-alkyl, aryl, heterocyclyl, heteroaryl, C₃₋₇-cycloalkyl, alkylenecycloalkyl, alkylenearyl, alkyleneheterocyclyl, alkyleneheteroaryl, C₂₋₆-alkenyl, C₂₋₆-alkynyl, C₁₋₄-alkoxy-C₁₋₅-alkyl radical, where 1-2 atoms in saturated carbocyclic radicals can also be replaced by heteroatoms, preferably N, O, or S, and up to 2 double bonds can be contained.
y=0, 1
Wₓ:
is —(CR_{w}⁴R_{w}⁵)_{w}—N R_{w}¹SO₂NR_{w}² R_{w}³
where w=0-3
R_{w}¹ is hydrogen, a branched or unbranched, optionally substituted C₁-C₆-alkyl, C₁-C₆-alkoxyalkyl, alkylenearyl, alkylenealkynyl, hetaryl, CO—C₁-C₆-alkyl, CO—O—C₁-C₆-alkyl or SO₂—C₁-C₆-alkyl radical or an optionally substituted C₃-C₇-cycloalkyl, CO—O-alkylenearyl, CO-alkylenearyl, CO-aryl, SO₂-aryl, CO-hetaryl or SO₂-alkylenearyl radical,
R_{w}², R_{w}³ independently of one another are hydrogen, a hydroxyl group, a branched or unbranched, optionally substituted C₁-C₈-alkyl, C₂-C₆-alkenyl, C₂-C₆-alkynyl or alkylenecycloalkyl radical, an optionally substituted C₃-C₈-cycloalkyl, aryl, arylalkyl, hetaryl or hetarylalkyl radical, or independently of one another both radicals R_{w}² and R_{w}³ together are a 3- to 7-membered, optionally substituted, saturated or unsaturated carbo- or heterocycle, which can contain up to three heteroatoms from the group consisting of O, N and S,
R_{w}⁴, R_{w}⁵ independently of one another are C₁₋₈-alkyl, halogen, OH, C₁₋₈-alkoxy,
R₁, R₂ independently of one another are hydrogen, halogen, CF₃, CN, NO₂, branched or unbranched C₁₋₈-alkyl, C₃₋₇-cycloalkyl, alkylcycloalkyl, where in each case 1-2 atoms in the cycloalkyl moiety can be replaced by N, O or S and up to 2 double bonds can be contained, aryl, alkylenearyl, hetaryl, alkylenehetaryl, C₂₋₆-alkenyl, C₃₋₆-alkynyl, C₀₋₄-alkyl-OR₃, C₀₋₄-alkyl-SR₃, SO—R₃, SO₂—R₃, C₀₋₄(CO)OR₃, O(CO)R₃, O(CO)NR₄R₅, C₀₋₄-alkyl-SO₂NR₄R₅, C₀₋₄-(CO)NR₄R₅, C₀₋₄-alkyl-NR₄R₅, CO—R₃,
or R₁ and R₂ together are a 3- to 9-membered optionally substituted cyclic or polycyclic system, which can contain 0-4 heteroatoms from the group consisting of O, N and S,
R₃ is H, or C₁₋₈-alkyl, aryl, heterocyclyl, heteroaryl, C₃₋₇-cycloalkyl, alkylenecycloalkyl, alkylenearyl, alkyleneheterocyclyl, alkyleneheteroaryl, C₂₋₆-alkenyl, C₂₋₆-alkynyl, C₁₋₄-alkoxy-C₁₋₅-alkylene, mono- or bisalkylaminoalkylene, acylaminoalkylene, each of which is branched or straight-chain and optionally substituted by halogen, OH, alkoxy, CN, COOH, COOC₁₋₄-alkyl, where in saturated carbocyclic radicals 1-2 atoms can also be replaced by heteroatoms, preferably N, O, or S, and up to 2 double bonds can be contained,
R₄, R₅ are H, C₁₋₈-alkyl, aryl, heterocyclyl, heteroaryl, C₃₋₇-cycloalkyl, alkylenecycloalkyl, alkylenearyl, alkyleneheterocyclyl, alkyleneheteroaryl, C₂₋₆-alkenyl, C₂₋₆-alkynyl, C₁₋₄-alkoxy-C₁₋₅-alkylene, mono- and bisalkylaminoalkylene, acylaminoalkylene, each of which is branched or straight-chain and optionally substituted by halogen, OH, alkoxy, CN, COOH, COOC₁₋₄-alkyl, where in saturated carbocyclic radicals 1-2 atoms can also be replaced by heteroatoms, preferably N, O, or S, and up to 2 double bonds can be contained,
A is a structural element selected from the group consisting of:
a 4- to 8-membered monocyclic saturated, unsaturated or aromatic hydrocarbon, which can contain up to 4 heteroatoms, selected from the group consisting of O, N and S, where in each case independently of one another the ring nitrogen optionally contained or the carbons can be substituted,
with the proviso that at least one heteroatom, selected from the group consisting of O, N and S, is contained in the structural element A, or
a 9- to 14-membered polycyclic saturated, unsaturated or aromatic hydrocarbon, which can contain up to 6 heteroatoms, selected from the group consisting of N, O and S, where in each case independently of one another the ring nitrogen optionally contained or the carbons can be substituted,
with the proviso that at least one heteroatom, selected from the group consisting of O, N and S, is contained in the structural element A,
a radical 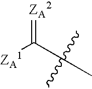
where
Z_{A}¹ is oxygen, sulfur or optionally substituted nitrogen, and
Z_{A}² is optionally substituted nitrogen, oxygen or sulfur,
or a radical 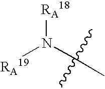
where
R_{A}¹⁸, R_{A}¹⁹
independently of one another are hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- or bisalkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical, or a radical —SO₂—R₄, —CO—OR₄, —CO—NR₄R₄ or —CO—R₄,
E is a spacer between A and the structural element pyrimidinone having 3-12 bonds.

2. A compound as claimed in claim 1, wherein the structural element A used is a structural element selected from the group consisting of the structural elements of the formulae I_{A}¹ to I_{A}¹⁹, 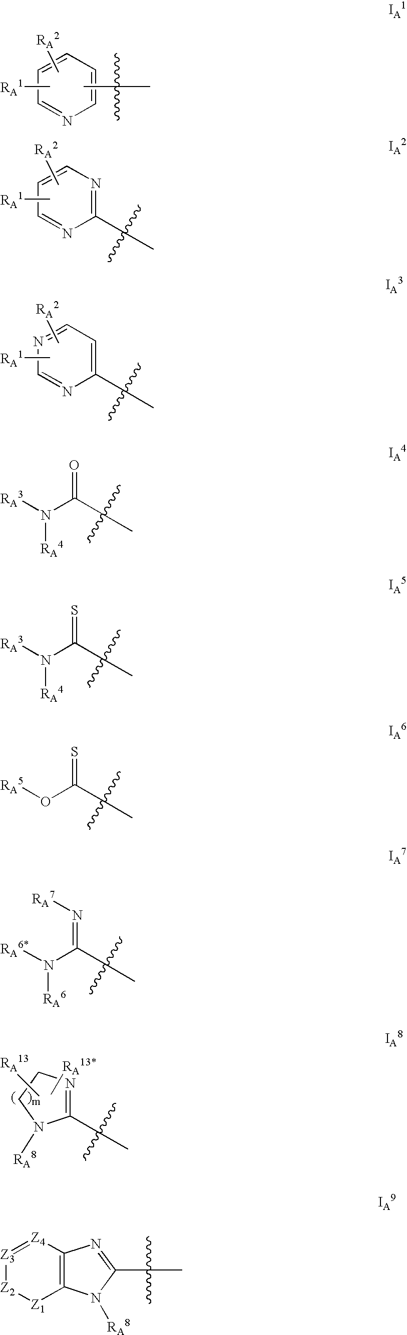 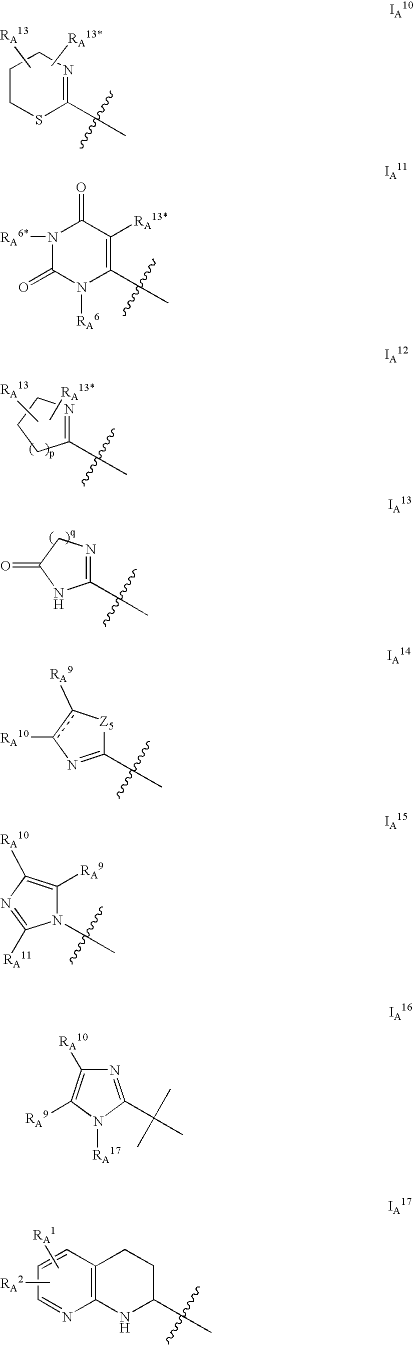 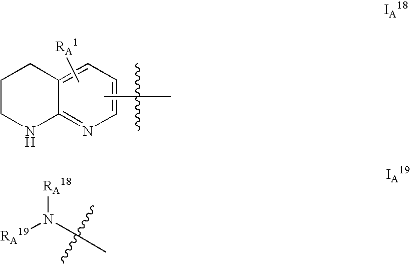
where
m, p, q independently of one another are 1, 2 or 3,
R_{A}¹, R_{A}² independently of one another are hydrogen, CN, halogen, a branched or unbranched, optionally substituted C₁-C₆-alkyl or CO—C₁-C₆-alkyl radical or an optionally substituted aryl, arylalkyl, hetaryl, hetarylalkyl or C₃-C₇-cycloalkyl radical or a radical CO—O—R_{A}¹⁴, O—R_{A}¹⁴, S—R_{A}¹⁴, NR_{A}¹⁵R_{A}¹⁶, CO—NR_{A}¹⁵R_{A}¹⁶ or SO₂NR_{A}¹⁵R_{A}¹⁶ or both radicals R_{A}¹ and R_{A}² together are a fused, optionally substituted, 5- or 6-membered, unsaturated or aromatic carbocycle or heterocycle which can contain up to three heteroatoms, selected from the group consisting of O, N, and S,
R_{A}¹³, R_{A}^{13*} independently of one another are hydrogen, CN, halogen, a branched or unbranched, optionally substituted C₁-C₆-alkyl radical or an optionally substituted aryl, arylalkyl, hetaryl or C₃-C₇-cycloalkyl radical or a radical CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}¹⁵R_{A}¹⁶,
where
R_{A}¹⁴ is hydrogen, a branched or unbranched, optionally substituted C₁-C₆-alkyl, alkylene-C₁-C₄-alkoxy, C₂-C₆-alkenyl, C₂-C₆-alkynyl or C₁-C₆-alkylene-C₃-C₇-cycloalkyl radical or an optionally substituted C₃-C₇-cycloalkyl, aryl, arylalkyl, hetaryl or hetarylalkyl radical,
R_{A}¹⁵, R_{A}¹⁶ independently of one another are hydrogen, a branched or unbranched, optionally substituted C₁-C₆-alkyl, CO—C₁-C₆-alkyl, SO₂—C₁-C₆-alkyl, COO—C₁-C₆-alkyl, CO—NH—C₁-C₆-alkyl, arylalkyl, COO-alkylenearyl, SO₂-alkylenearyl, CO—NH-alkylenearyl, CO—NH-alkylenehetaryl or hetarylalkyl radical or an optionally substituted C₃-C₇-cycloalkyl, aryl, CO-aryl, CO—NH-aryl, SO₂-aryl, hetaryl, CO—NH-hetaryl, or CO-hetaryl radical,
R_{A}³, R_{A}⁴ independently of one another are hydrogen, —(CH₂)ₙ—(X_{A})ⱼ—R_{A}¹², or both radicals together are a 3- to 8-membered, saturated, unsaturated or aromatic N heterocycle which additionally can contain two further, identical or different heteroatoms O, N, or S, where the cycle can be optionally substituted or a further, optionally substituted, saturated, unsaturated or aromatic cycle can be fused to this cycle,
where
n is 0, 1, 2 or 3,
j is 0 or 1,
X_{A} is —CO—, —CO—N(R_{L}¹)—, —N(R_{L}¹)—CO—, —N(R_{L}¹)—CO—N(R_{L}¹*)—, —N(R_{L}¹)—CO—O—, —O—, —S—, —SO₂—, —SO₂—N(R_{L}¹)—, —SO₂—O—, —CO—O—, —O—CO—, —O—CO—N(R_{L}¹)—, —N(R_{L}¹)— or —N(R_{L}¹)—SO₂—,
R_{A}¹² is hydrogen, a branched or unbranched, optionally substituted C₁-C₆-alkyl radical, a C₂-C₆-alkynyl or C₂-C₆-alkenyl radical which is optionally substituted by C₁-C₄-alkyl or aryl, or a 3- to 6-membered, saturated or unsaturated heterocycle which is substituted by up to three identical or different radicals, which can contain up to three different or identical heteroatoms O, N, S, a C₃-C₇-cycloalkyl, aryl or heteroaryl radical, where two radicals together can be a fused, saturated, unsaturated or aromatic carbocycle or heterocycle, which can contain up to three different or identical heteroatoms O, N, S, and the cycle can be optionally substituted or a further, optionally substituted, saturated, unsaturated or aromatic cycle can be fused to this cycle, or the radical R_{A}¹², together with R_{L}¹ or R_{L}¹*, forms a saturated or unsaturated C₃-C₇-heterocycle, which can optionally contain up to two further heteroatoms, selected from the group consisting of O, S and N,
R_{L}¹, R_{L}¹* independently of one another are hydrogen, a branched or unbranched, optionally substituted C₁-C₆-alkyl, C₁-C₆-alkoxyalkyl, C₂-C₆-alkenyl, C₂-C₁₂-alkynyl, CO—C₁-C₆-alkyl, CO—O—C₁-C₆-alkyl or SO₂—C₁-C₆-alkyl radical or an optionally substituted C₃-C₇-cycloalkyl, aryl, arylalkyl, CO—O-alkylenearyl, CO-alkylenearyl, CO-aryl, SO₂-aryl, hetaryl, CO-hetaryl- or SO₂-alkylenearyl radical,
R_{A}⁵ is a branched or unbranched, optionally substituted C₁-C₆-alkyl, arylalkyl, C₃-C₇-cycloalkyl or C₁-C₄-alkylene-C₃-C₇-cycloalkyl radical or an optionally substituted aryl, hetaryl, heterocycloalkyl or heterocycloalkenyl radical,
R_{A}⁶, R_{A}^{6*} are hydrogen, a branched or unbranched, optionally substituted C₁-C₄-alkyl, —CO—O—C₁-C₄-alkyl, arylalkyl, —CO—O-alkylenearyl, —CO—O-allyl, —C₀-C₁-C₄-alkyl, —CO-alkylenearyl, C₃-C₇-cycloalkyl or —CO-allyl radical or both radicals R_{A}⁶ and R_{A}⁶ in structural element I_{A}⁷ together are an optionally substituted, saturated, unsaturated or aromatic heterocycle, which in addition to the ring nitrogen can contain up to two further different or identical heteroatoms O, N, S,
R_{A}⁷ is hydrogen, —OH, —CN, —CONH₂, a branched or unbranched, optionally substituted C₁-C₄-alkyl, C₁-C₄-alkoxy, C₃-C₇-cycloalkyl or —O—CO—C₁-C₄-alkyl radical, or an optionally substituted arylalkyl, —O-alkylenearyl, —O—CO-aryl, —O—CO-alkylenearyl or —O—CO-allyl radical, or both radicals R_{A}⁶ and R_{A}⁷ together are an optionally substituted, unsaturated or aromatic heterocycle, which in addition to the ring nitrogen can contain up to two further different or identical heteroatoms O, N, S,
R_{A}⁸ is hydrogen, a branched or unbranched, optionally substituted C₁-C₄-alkyl, CO—C₁-C₄-alkyl, SO₂—C₁-C₄-alkyl or CO—O—C₁-C₄-alkyl radical or an optionally substituted aryl, CO-aryl, SO₂-aryl, CO—O-aryl, CO-alkylenearyl, SO₂-alkylenearyl, CO—O-alkylenearyl or alkylenearyl radical,
R_{A}⁹, R_{A}¹⁰ independently of one another are hydrogen, —CN, halogen, a branched or unbranched, optionally substituted C₁-C₆-alkyl radical or an optionally substituted aryl, arylalkyl, hetaryl, C₃-C₇-cycloalkyl radical or a radical CO—O—R_{A}¹⁴, O—R_{A}¹⁴, S—R_{A}¹⁴, NR_{A}¹⁵R_{A}¹⁶, SO₂—NR_{A}¹⁵ R_{A}¹⁶ or CO—NR_{A} R_{A}, or both radicals R_{A}⁹ and R_{A}¹⁰ together in structural element I_{A}¹⁴ are a 5- to 7-membered saturated, unsaturated or aromatic carbocycle or heterocycle, which can contain up to three different or identical heteroatoms O, N, S and is optionally substituted by up to three identical or different radicals,
R_{A}¹¹ is hydrogen, —CN, halogen, a branched or unbranched, optionally substituted C₁-C₆-alkyl radical or an optionally substituted aryl, arylalkyl, hetaryl or C₃-C₇-cycloalkyl radical or a radical CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}¹⁵R_{A}¹⁶,
R_{A}¹⁷ is hydrogen or both radicals R_{A}⁹ and R_{A}¹⁷ in structural element I_{A}¹⁶ together are a 5- to 7-membered saturated, unsaturated or aromatic heterocycle, which in addition to the ring nitrogen can contain up to three different or identical heteroatoms O, N, S and is optionally substituted by up to three identical or different radicals,
R_{A}¹⁸, R_{A}¹⁹ independently of one another are hydrogen, a branched or unbranched, optionally substituted C₀-C₈-alkyl, C₂-C₆-alkenyl, C₂-C₆-alkynyl, C₁-C₅-alkylene-C₁-C₄-alkoxy, mono- or bisalkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkyleneheterocycloalkyl, C₁-C₄-alkyleneheterocycloalkenyl or hetarylalkyl radical, or a radical —SO₂—R⁴, —CO—OR⁴, —CO—NR⁴R⁴ or —CO—R⁴,
Z₁, Z₂, Z₃, Z₄ independently of one another are nitrogen, C—H, C-halogen or a branched or unbranched, optionally substituted C—C₁-C₄-alkyl or C—C₁-C₄-alkoxy radical
Z₅ is NR_{A}⁸, oxygen or sulfur

3. A compound as claimed in one of claims 1 or 2, wherein the spacer structural element E is a structural element of the formula I_{E}
(NR_{E}¹)ᵢ-E-(U_{E})_{g} (I_{E})
where
(NR_{E}¹)i is the A-terminal end and (U_{E})_{g} the pyrimidinone-terminal end of the spacer structural element E,
U_{E} is oxygen, sulfur or NR_{E}²,
g is 0 or 1,
i is 0 or 1,
R_{E}¹ and R_{E}² independently of one another are hydrogen, a branched or unbranched, optionally substituted C₁-C₆-alkyl, alkoxyalkyl, alkylenearyl, alkylenealkynyl, hetaryl, CO—C₁-C₆-alkyl, CO—O—C₁-C₆-alkyl or SO₂—C₁-C₆-alkyl radical or an optionally substituted C₃-C₇-cycloalkyl, CO—O-alkylenearyl, CO-alkylenearyl, CO-aryl, SO₂-aryl, CO-hetaryl or SO₂-alkylenearyl radical, SO₂-hetaryl, SO₂-alkylenehetaryl, and
E₁ is a structural element of the formula I_{E1}
—(CR_{E}³R_{E}⁴)ₖ₁-(L_{E})ₖ₂-(CR_{E}⁵R_{E}⁶)ₖ₃-(Q_{E})ₖ₄-(CR_{E}⁷R_{E}⁸)ₖ₅-(T_{E})ₖ₆-(CR_{E}⁹ R_{E}¹⁰)ₖ₇— I_{E1}
where
k2, k4, k6 are 0 or 1,
k1, k3, k5, k7 are 0, 1 or 2,
R_{E}³, R_{E}⁴, R_{E}⁵, R_{E}⁶, R_{E}⁷, R_{E}⁸, R_{E}⁹, R_{E}¹⁰ independently of one another are hydrogen, halogen, a hydroxyl group, a branched or unbranched, optionally substituted C₁-C₆-alkyl, C₂-C₆-alkenyl, C₂-C₆-alkynyl or alkylenecycloalkyl radical, a radical —(CH₂)_{X}-(Y_{E})_{Z}-R_{E}¹¹, an optionally substituted C₃-C₇-cycloalkyl, aryl, arylalkyl, hetaryl or hetarylalkyl radical or independently of one another in each case two radicals R_{E}³ and R_{E}⁴ or R_{E}⁵ and R_{E}⁶ or R_{E}⁷ and R_{E}⁸ or R_{E}⁹ and R_{E}¹⁰ together are a 3- to 7-membered, optionally substituted, saturated or unsaturated carbo- or heterocycle, which can contain up to three heteroatoms from the group consisting of O, N and S,
x is 0, 1, 2, 3 or 4,
z is 0 or 1,
Y_{E} is —CO—, —CO—N(R_{y}²)—, —N(R_{y}²)—CO—, —N(R_{y}²)—CO—N(R_{y}²*)—, —N(R_{y}²)—COO—, —O—, —S—, —SO₂—, —SO₂—N(R_{y}²)—, —SO₂—O—, —CO—O—, —O—CO—, —O—CO—N(R_{y}²)—, —N(R_{y}²)— or —N(R_{y}²)—SO₂—,
R_{y}², R_{y}²* independently of one another are hydrogen, a branched or unbranched, optionally substituted C₁-C₆-alkyl, C₂-C₆-alkenyl, C₂-C₈-alkynyl, CO—C₁-C₆-alkyl, CO—O—C₁-C₆-alkyl or SO₂—C₁-C₆-alkyl radical or an optionally substituted hetaryl, hetarylalkyl, arylalkyl, C₃-C₇-cycloalkyl, CO—O-alkylenearyl, CO-alkylenearyl, CO-aryl, SO₂-aryl, CO-hetaryl or SO₂-alkylenearyl radical,
R_{E}¹¹ is hydrogen, a hydroxyl group, CN, halogen, a branched or unbranched, optionally substituted C₁-C₆-alkyl radical, an optionally substituted C₃-C₇-cycloalkyl, aryl, heteroaryl or arylalkyl radical, a C₂-C₆-alkynyl or C₂-C₆-alkenyl radical optionally substituted by C₁-C₄-alkyl or aryl, an optionally substituted C₆-C₁₂-bicycloalkyl, C₁-C₆-alkylene-C₆-C₁₂-bicycloalkyl, C₇-C₂₀-tricycloalkyl or C₁-C₆-alkylene-C₇-C₂₀-tricycloalkyl radical, or a 3- to 8-membered, saturated or unsaturated heterocycle substituted by up to three identical or different radicals, which can contain up to three different or identical heteroatoms O, N, S, where two radicals together can be a fused, saturated, unsaturated or aromatic carbocycle or heterocycle, which can contain up to three different or identical heteroatoms O, N, S, and the cycle can be optionally substituted or a further, optionally substituted, saturated, unsaturated or aromatic cycle can be fused to this cycle, or the radical R_{E}¹¹ together with R_{y}² or R_{y}²* forms a saturated or unsaturated C₃-C₇-heterocycle, which optionally can contain up to two further heteroatoms, selected from the group consisting of O, S and N,
L_{E}, T_{E} independently of one another are CO, CO—NR_{E}¹², NR_{E}¹²—CO, sulfur, SO, SO₂, SO₂—NR_{E}¹², NR_{E}¹²—SO₂, CS, CS—NR_{E}¹², NR_{E}¹²—CS, CS—O, O—CS, CO—O, O—CO, oxygen, ethynylene, CR_{E}¹³—O—CR_{E}¹⁴, C(═CR_{E}¹³R_{E}¹⁴), CR_{E}¹³═CR_{E}¹⁴, —CR_{E}¹³(OR_{E}¹⁵)—CHR_{E}¹⁴, —CHR_{E}¹³—CR_{E}¹⁴(OR_{E}¹⁵)—,
R_{E}¹² is hydrogen, a branched or unbranched, optionally substituted C₁-C₆-alkyl, C₂-C₆-alkenyl, C₂-C₈-alkynyl or an optionally substituted C₃-C₇-cycloalkyl, hetaryl, arylalkyl or hetarylalkyl radical or a radical CO—R_{E}¹⁶, COOR_{E}¹⁶ or SO₂-R_{E}¹⁶,
R_{E}¹³ R_{E}¹⁴ independently of one another are hydrogen, a hydroxyl group, a branched or unbranched, optionally substituted C₁-C₆-alkyl, C₁-C₄-alkoxy, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,
R_{E}¹⁵ is hydrogen, a branched or unbranched, optionally substituted C₁-C₆-alkyl, C₂-C₆-alkenyl, C₂-C₆-alkynyl or alkylenecycloalkyl radical or an optionally substituted C₃-C₇-cycloalkyl, aryl, arylalkyl, hetaryl or hetarylalkyl radical,
R_{E}¹⁶ is hydrogen, a hydroxyl group, a branched or unbranched, optionally substituted C₁-C₆-alkyl, C₂-C₆-alkenyl, C₂-C₆-alkynyl or C₁-C₅-alkylene-C₁-C₄-alkoxy radical, or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-C₃-C₇-hetero-cycloalkyl, C₁-C₄-alkylene-C₃-C₇-heterocycloalkenyl or hetarylalkyl radical and
Q_{E} is an optionally substituted 4- to 11-membered mono- or polycyclic, aliphatic or aromatic hydrocarbon, which can contain up to 6 double bonds and up to 6 identical or different heteroatoms, selected from the group consisting of N, O and S, where the ring carbons or ring nitrogens can be optionally substituted.

4. A pharmaceutical preparation comprising at least one compound as claimed in one of claims 1 to 3 and customary excipients and/or vehicles.

5. The use of the compounds as claimed in at least one of claims 1 to 3 for the treatment of diseases in which the interaction between integrins and their natural ligands is excessive or reduced.

6. A procedure for the treatment and/or prophylaxis of diseases in which the interaction between integrins and their natural ligands is excessive or reduced, by administering an efficacious amount of at least one compound as claimed in one of claims 1 to 3.
